(19) United States
(12) Patent Application Publication
(10) Pub. No.: US 2013/0109616 A1
(43) Pub. Date: May 2, 2013

(54) ANTIMICROBIAL PEPTIDES

(71) Applicants: William Eldridge, Essex (GB); Christopher Ullman, Essex (GB); Marc Alan Fox, Salisbury (GB); David Okhono Ulaeto, Salisbury (GB); Joanne Elizabeth Thwaite, Salisbury (GB)

(72) Inventors: William Eldridge, Essex (GB); Christopher Ullman, Essex (GB); Marc Alan Fox, Salisbury (GB); David Okhono Ulaeto, Salisbury (GB); Joanne Elizabeth Thwaite, Salisbury (GB)

(73) Assignee: ISOGENICA LTD, Essex (GB)

(21) Appl. No.: 13/521,457

(22) PCT Filed: Jan. 11, 2011
(86) PCT No.: PCT/GB11/50036

§ 371 Date: Jan. 7, 2013

(30) Foreign Application Priority Data
Jan. 11, 2010 (GB) 1000352.3

(51) Int. Cl. C07K 14/435

(52) U.S. Cl. CPC C07K 14/435
USPC 514/2.8; 530/324; 530/326; 536/23.5; 514/2.3; 514/2.4

(57) ABSTRACT

A method is provided for isolating protease resistant antimicrobial peptides (AMPs) from a peptide display library. A plurality of nucleic acid constructs that encode displayed peptides are expressed, resulting in the formation of a plurality of peptide-nucleic acid complexes, each complex comprising at least one displayed peptide associated with the corresponding nucleic acid construct encoding the displayed peptide. The complexes are exposed to at least one protease, to allow the proteolysis of protease-sensitive peptides, such that resistant peptides remain. The peptide-nucleic acid complexes are further exposed to a membrane composition to allow association of complexes that contain membrane-associating peptides. Complexes that remain unassociated with the membrane are removed; and membrane-associated complexes are recovered. The AMPs so characterised may be resistant to one or more protease enzymes and exhibit antimicrobial activity against one or more microbe.

### FIELD OF THE INVENTION

This invention relates to methods for the isolation of novel molecules termed antimicrobial peptides (AMPs). In particular, the AMPS are characterised by their improved proteolytic resistance to proteolytic enzymes.

### BACKGROUND OF THE INVENTION

Antimicrobial peptides (AMPs) are produced by a broad range of different organisms as part of an innate-immune response, and may demonstrate antimicrobial activity against bacteria (gram-positive and gram-negative), fungi, parasites and enveloped viruses. Typically, AMPs are positively charged (e.g. having a net charge between +2 and +9), are between 12 and 100 amino acids in length, and form an amphiphilic structure (for reviews see: Zasloff, 2002; Jenssen et al., 2006; Zaiou, 2007; Bechinger, 2008; and Namjoshi et al., 2008).

AMPs can play two main roles in response to microbial invasion of the host: (i) directly attacking the microbe (for example, via killing, inactivation of virulence factors, or blunting biological activity e.g. by binding to toxins); or (ii) as mediators of host immune responses (see e.g. Bals et al., 1999; Hancock & Diamond, 2000; and Bowdish et al., 2004).

LL-37, a 37 amino acid peptide fragment of the human cationic antimicrobial protein 18 (hCAP18), is the only known human cathelicidin (Gudmondsson et al., 1996). It has been reported to have both direct antimicrobial activity and indirect activity as an initiator of host response (for a review see Durr et al., 2006). However, previous studies have shown that LL-37 is susceptible to degradation by proteases secreted by various bacteria, such as Bacillus species (Thwaite at al., 2006), Streptococci (Johansson et al., 2008) and Staphylococci (Sieprawska-Lupa et al., 2004). Unfortunately, this degradation is known to inactivate LL-37 and significantly limits its use as a therapeutic.

The structure and activity of LL-37 has been studied in detail, and it is a core region of the peptide that has been shown to be necessary for antimicrobial activity. The core region, which is structured in SDS micelles (Li et al., 2006), encompasses amino acids 17 to 29. Fragments of LL-37 that include this core region, such as the amino acid fragment of 17 to 37, are known to have equivalent bactericidal activity to the complete 37 amino acid peptide. However, this region is known to be susceptible to the activity of bacterial proteases, such as aureolysin (Schmidtchen et al., 2002; and Sieprawska-Lupa et al., 2004). For example: an analysis of the degradation of LL-37 by S. aeruginosa elastase identified cleavage sites at Asn30-Leu31, Asp26-Phe27, Arg12-Ile13, Arg19-Ile20, Arg23-Ile24, Glu16-Phe17; cleavage sites for aureolysin at Arg19-Ile20, Arg23-Ile24, Leu31-Val32; and a cleavage site for V8 protease at Glu16-Phe17. Rational design approaches have sought to address the problem of protease degradation by stabilising specific cleavage sites. Thus, Strömstedt et al., 2009 reported the stabilisation of a 17 amino acid region of LL-37 known as EFK-17 through the replacement of Phe17, Ile20, Ile24 and Phe27 with D-enantiomers, or through substitution with tryptophan. However, these tryptophan substitutions did not protect against V8 protease and aureolysin and significantly, none of the mutated peptides was as active as the parental LL-37 molecule in its antimicrobial activity.

Therefore, there is a need in the art for AMPs that have increased resistance to degradation or inactivation by proteases. Particularly, there is a need for AMPs for use in humans, which have increased resistance to degradation or inactivation by proteases. Furthermore, it would be desirable to have such proteolytic-resistant AMPs that retain sufficient antimicrobial activity to be useful against microbial infections, such as in humans. More desirably, it would be useful to have such AMPs that have as much, or even more activity than a corresponding wild-type AMP. It would be useful to have proteolytic-resistant AMPs that are active against bacteria (e.g. gram-positive and/or gram-negative), fungi, parasites and/or enveloped viruses; particularly of human and animal microbes. Furthermore, it would be desirable to have methods for designing and selecting such proteolytic-resistant AMPs.

Accordingly, the present invention seeks to overcome or at least alleviate one or more of the problems in the prior art.

### SUMMARY OF THE INVENTION

In general, the present invention provides novel antimicrobial peptides (AMPs) that demonstrate increased resistance to at least one proteolytic enzyme. These AMPs also exhibit antimicrobial activity against at least one microbe. Furthermore, the invention relates to methods for the selection of such novel AMPs that have one or more desirable activity, such as increased resistance to one or more proteolytic enzyme and, desirably, antimicrobial activity against one or more microbe. Thus, via the methods of the invention, AMPs are selected for their ability to resist degradation by one or more proteases. In addition, selected peptides are characterised by their ability to inhibit or kill one or more microbe. In particular, the novel AMPs are selected to show improved characteristics over LL-37.

Hence, in this invention, the Applicant has created libraries based upon the natural sequence of LL-37 and a fragment of this peptide, and subjected these libraries to a selection process to isolate the most proteolytically stable peptides. In some examples, the peptides were selected in the presence of bacterial proteases selected from V8 protease and subtulisin so that non-resistant peptides would be degraded and only those peptide sequences that are most resistant surviving the selection. In addition, selection of the most resistant peptides against bacterial membranes can be used to maintain a positive selection pressure for biological activity. In this way, novel AMPs have been identified that have antimicrobial activity and that have proteolytic resistance that is improved over the LL-37 peptide. Furthermore, the novel AMPs are interesting because, compared to the wild-type sequence, they include mutations that are outside the known enzyme substrate sites in the wild-type sequence of the LL-37 peptide, and provide a shorter peptide that confers resistance to proteases.

Accordingly, in a first aspect of the invention there is provided a method for isolating a protease resistant antimicrobial peptide from a peptide display library, the library comprising a plurality of nucleic acid sequences that encode displayed peptides, comprising the steps of: (a) expressing a plurality of nucleic acid constructs, wherein each nucleic acid construct comprises a promoter sequence operably linked to the nucleic acid sequence, such that expression of the plurality of nucleic acid constructs results in formation of a plurality of peptide-nucleic acid complexes, each complex comprising at least one displayed peptide associated with the corresponding nucleic acid construct encoding the displayed peptide; (b) exposing the plurality of peptide-nucleic acid complexes to at least one protease, and allowing proteolysis of protease-sensitive peptides to occur; (c) exposing the peptide-nucleic acid complexes to a membrane composition; (d) removing any peptide-nucleic acid complexes that remain unassociated with membranes of the membrane composition; and (e) recovering any membrane-associated nucleic acid-peptide complexes, and characterising the peptide encoded by the nucleic acid sequence of any membrane-associated complex as comprising a protease-resistant antimicrobial peptide (AMP). Generally, in accordance with the invention, step (b) is carried out prior to step (c). However, in some circumstances it may be desirable to reverse the order of steps (b) and (c). The above method represents a round of selection for identifying AMPs having desirable properties, such as improved resistance to proteases and/or increased anti-microbial activity. One or more (e.g. 2, 3, 4, 5, 6 or more) rounds of selection may be used in accordance with the invention as desired.

Suitably, the membrane composition is a bacterial membrane composition. Conveniently, the bacteria are from the species Bacillus, and particularly, the membrane is from Bacillus subtilis. Alternatively, the membrane composition may be derived from a bacterial species such as Clostridium, Saphylococcus, Mycobacterium or Escherichia. Particularly suitable additional membrane compositions for use in accordance with the invention are obtained from Clostridium difficile, Staphlococcus aureus, Mycobacterium tuberculosis and Escherichia coli. In another embodiment, the membrane may be derived from a yeast, in particular Candida. In this way, the membrane can advantageously be derived from a species or strain of microbe that is the same as, or is a convenient substitute for, the microbe against which the AMP is intended to have antimicrobial activity. Where it is not convenient to use a microbial membrane composition, the membrane composition may be an artificial membrane composition or lipid mono- or bilayer; for example, a population of lipid-encapsulated compartments, such as micelles or liposomes, or fragments thereof. In some embodiments, intact membranes (e.g. from intact cells, such as bacterial cells) may be used in place of membrane fragments.

It will be appreciated that any desirable protease enzyme or enzymes may be used in step (b) in order that non-resistant peptides (or at least the most protease-sensitive peptides) are degraded by the protease(s) and removed from the pool of potential AMPs. The one or more protease for use in this step is typically selected according to which proteases it is wished to select for resistance against. Thus, the protease may be an animal or human protease enzyme (such as those found in the blood of the animal concerned), for example, chymotrypsin and/or trypsin. In some embodiments both chymotrypsin and trypsin are used. Alternatively, the protease may be one or more that is produced by a microbe, such as a bacterium, fungus, virus or parasite against which the AMP is to be directed. By way of example, the protease may be selected from subtilisin and/or V8 protease and/or Entamoeba histolytica cysteine protease. In some embodiments both subtilisin and V8 protease may be used. Advantageously, for example, where it is desirable to confer multiple protease resistance to the AMPs more than one, such as 2, 3 or 4 proteases may be used. Accordingly, the method may comprise exposing the peptide-nucleic acid complexes to a combination of chymotrypsin, trypsin, subtilisin and V8 protease. In other embodiments, the method may comprise exposing the peptide-nucleic acid complexes to a combination of chymotrypsin, trypsin and Entamoeba histolytica cysteine protease. Where the method includes more than one round of selection, the peptide-nucleic acid complexes may be exposed to one or more of the proteases (e.g. chymotrypsin and trypsin) in one round of selection, and to one or more of the same or different proteases (e.g. chymotrypsin, trypsin, subtilisin and V8 protease) in another round of selection. Subtilisin and/or V8 protease may be used in a round of selection subsequent to a round in which chymotrypsin and/or trypsin are used or vice versa. Similarly, Entamoeba histolytica cysteine protease may be used in a different round of selection to a round in which chymotrypsin and/or trypsin are used.

In some embodiments, it may be convenient to couple at least one protease to a solid support, such as a tube, plate or agarose bead, for ease of handling and/or for ease of separating protease(s) from the peptide-nucleic acid complexes.

The length of time for which the peptide-nucleic acid complexes are exposed to the protease(s), and the quantity and activity of the protease(s) used is selected according to requirements. For example, the amount of a protease enzyme may be increased and/or the incubation time increased to help select for peptides having increased resistance to the protease.

Peptide-nucleic acid complexes may be separated from protease(s) using any convenient means, for example, magnetic separation, affinity separation and/or centrifugation. A particularly convenient means is centrifugation, in which the peptide-nucleic acid complexes remain in solution while the heavy protease-containing particles (e.g. beads) are pelleted. Alternatively, the step of exposing the peptide-nucleic acid complexes to the one or more protease may be carried out following or during the step of exposing the peptide-nucleic acid complexes to the membrane composition. The proteases can be removed by a convenient means such as washing and/or centrifugation. Alternatively, the protease activity may be removed by the addition of protease inhibitors such as Pefabloc (4-(2-Aminoethyl)benzenesulfonyl fluoride hydrochloride) or PMSF (phenylmethanesulphonylfluoride) or E-64 (trans-epoxysuccinyl-I-leucylamido-(4-guanidino)butane).

Protease degradation of a peptide causes the separation of peptide-nucleic acid complexes and prevents the peptide-nucleic acid complex from associating with the membrane composition of step (c).

As with step (b) above, in step (d) peptide-nucleic acid complexes that remain unassociated with membranes may be separated from membrane-associated complexes using any suitable system. In some embodiments the membranes may be immobilised on a solid surface, such as a tube, plate or bead (e.g. polystyrene or magnetic beads). One convenient method of separating unassociated complexes from membrane-associated complexes is by centrifugation, which can be used to pellet the membranes and the associated complexes. Typically, one or more washing steps (with a suitable buffer) may be used to remove unassociated peptide-nucleic acid complexes from the membrane compositions. It will be appreciated that different types of buffers (e.g. higher or lower salt concentrations and different quantities or types of detergent) may be used to aid in the selection of membrane associated complexes, for example, by removing non-associated or weakly-associated complexes, as desired, so that only the more strongly associated complexes are retained. This procedure may also be used to remove proteases from the selection mixture, as previously indicated.

As described in our co-pending patent application, WO2007/010293, the selection method is conveniently based on an in vitro peptide display library. Most suitably, the method comprises a CIS in vitro peptide display library, which provides a number of benefits, including the large library size that can be used and the avoidance of any in vivo steps, such as the sub-cloning of selected nucleic acids for further rounds of selection and maturation.

The methods of the invention may further comprise correlating the one or more expressed AMPs of step (e) with the corresponding nucleic acid constructs, thereby identifying nucleic acid sequences for the AMPs. Any such nucleic acid sequence can be mutated taking into account known codon redundancy, to generate mutated nucleic acid molecules that encode the same peptide sequences. Alternatively, the nucleic acid molecules identified can be mutated by substitution, deletion or addition to encode peptide sequences having 1, 2, 3, 4, 5 or more amino acid changes to those of the selected peptides. Such modifications may be generated via a designed mutagenesis procedure or by using a maturation process, for example, by creating a sub-library of nucleic acid molecules based on the identified nucleic acid, and selecting for peptides having particularly desirable or improved properties over the originally selected peptide.

Typically, in aspects and embodiments of the invention, the AMP comprises an amino acid sequence of between 12 and 50 residues, such as between 15 and 45 residues, suitably between 18 and 40 residues or between 18 and 37 residues, and more suitably between 20 and 37 residues in length. In some cases the AMP comprises a sequence of 20 to 24 amino acids. For ease of expression, the peptide may include an N-terminal leader amino acid sequence, e.g. of 1, 2, 3, 4, 5 or more amino acids, such as the sequence Met-Ala (MA), which are expressed in front of the AMP sequence. The leader peptide may aid in the cloning or expression of the peptide.

Suitably, the method further comprises isolating an AMP. Thus, in accordance with a second aspect of the invention, there is provided an AMP peptide as may be identified by the methods of the invention. More suitably, the AMP is an isolated peptide. The invention further encompasses derivatives of the AMPs of the invention, and therefore, the method may comprise the step of forming (e.g. making or selecting) a derivative of an AMP of the invention. Any such derivative may be or comprise an AMP of the invention and is, therefore, within the scope of the invention. According to the methods of the invention, the derivative may be formed by modifying the AMP by performing a maturation experiment to improve one or more characteristics of the AMP. The person of skill in the art can readily devise and identify such maturation experiments. A “matured” AMP (i.e. a derivative) typically may comprise 1, 2, 3, 4, 5 or more amino acid modifications (deletions, substitutions, additions) to the original sequence; more typically, a derivatised AMP comprises 1, 2 or 3 modifications.

Conveniently, the AMP on the invention is based on the wild-type LL-37 peptide (LLGDFFRKSKEKIGKEFKRIVQRIKDFLRNLVPRTES; SEQ ID NO: 1) or a fragment thereof having at least one sequence mutation (such as a deletion, addition or substitution). Typically, the mutation is one or more amino acid substitution. In one beneficial embodiment, the sequence of wild-type LL-37 (or a fragment thereof) is mutated to increase its cationic charge.

In some embodiments wherein the AMP is derived from the sequence of LL-37, the AMP of the invention includes one or more mutation selected from the group of: Ser37 to Asn, Gly, Cys, Arg, Thr; Glu36 to Lys, Met, Gln, Glu, Ile; Thr35 to Ile, Ser, Arg, Lys, Ala, Asn, Ile, Cys or Pro; Arg34 to Cys or Gly; Pro33 to Met or Ser; Val32 to Met; Asn30 to Lys, Ile or Tyr; Arg29 to His or Leu; Leu28 to Arg; Phe27 to Val or Leu; Asp26 to Val or Tyr; Lys25 to Glu; Ile24 to Ser, Leu or Met; Arg23 to His; GIn22 to Leu, Arg or His; Val21 to Leu or Ala; Lys18 to Arg, Glu or Gln; Phe17 to Val, Ile, Leu or Cys; Glu16 to Lys, Asn or Ile; Lys15 to Arg or Glu; Gly14 to Val; Ile13 to Phe, Thr or Met; Lys12 to Gly or Met; Glu11 to Ala or Val; Lys10 to Arg or Gln; Ser9 to Arg, Gly or Cys; Lys8 to Glu, Gln or Met; Arg7 to His, Ser or Leu; Phe6 to Val; Phe5 to Val; Asp4 to Val, Asn or Glu; Gly3 to Val.

Particularly suitable AMPs of the invention comprise at least 12 amino acid residues of the sequence of LL-37, wherein the AMP comprises at least 3 amino acid mutations of the sequence of LL-37. For example, the AMP of the invention may comprise 18 amino acids of the sequence FKRIVQRIKDFLRNLVPRTES with 3 or more of these amino acid residues mutated to a different (non-wild-type) residue. In these embodiments, the at least 3 mutations are suitably selected from the substitutions specified above.

In one suitable embodiment, the 3 or more substitutions may be selected from the group consisting of: Ser37 to Asn; Glu36 to Lys, Arg or Met; Thr35 to Ile, Ser, Lys, Arg or Cys; Pro33 to Ser, Met or Ala; Val32 to Met; Asn30 to Lys or Ile; Leu28 to Arg; Asp26 to Val or Cys; Val21 to Leu; Lys18 to Arg; Phe17 to Val, Ile or Leu; Glu16 to Lys; Glu11 to Ala; Ser9 to Arg; and Asp4 to Val. More suitably, the at least 3 substitutions are selected from: Ser37 to Asn; Glu36 to Lys, Arg or Met; Thr35 to Ile, Ser, Lys, Arg or Cys; Pro33 to Ser, Met or Ala; Val32 to Met; and Asn30 to Lys or Ile.

In a particularly suitable embodiment the AMP sequence comprises at least 18 consecutive amino acids of the sequence: N′-R I V Q R I K X₁ F X₂ R X₃ L X₄ X₅ R X₆ X₇ X₈-C′; wherein X₁ is D, V, C or Y; X₂ is L or R; X₃ is N, I or K; X₄ is V or M; X₅ is P, S, A or M; X₆ is T, K, R, C, I or S; X₇ is E, K, R or M; and X₈ is S or N. More suitably, X₁ is D or V; X₂ is L or R; X₃ is N or K; X₄ is V or M; X₅ is P, S or M; X₆ is K, R, S or I; X₇ is K, R or M; and X₈ is S or N. Still more suitably, X₁ is D; X₂ is L; X₅ is P or M; X₆ is R, S or I; and X₇ is K.

More particularly, the AMP may be selected from an LL-37 peptide or fragment comprising at least the following amino acid substitutions: (i) Asp4 to Val, Ser9 to Arg, Glu11 to Ala, Glu16 to Lys, Val21 to Ile, Leu28 to Arg, Thr35 to Ile and Glu36 to Lys; (ii) Phe17 to Val, Asp26 to Val, Val32 to Met, Thr35 to Ser and Glu36 to Lys; (iii) Lys18 to Arg, Pro33 to Met, Thr35 to Arg, Glu36 to Lys and Ser37 to Asn; or (iv) Asn30 to Lys, Thr35 to Ile and Glu36 to Met. Preferably, the AMP comprises between 18 and 37 consecutive amino acids of the mutated sequence of LL-37.

In another embodiment, the AMP comprises: (i) the sequence of RIVQRIKDFLR of LL-37; and (ii) at least 3 amino acid substitutions selected from Ser37 to Asn; Glu36 to Lys, Arg or Met; Thr35 to Ile, Ser or Arg; Pro33 to Met; Val32 to Met; and Asn30 to Lys of LL-37. In yet another embodiment, the AMP comprises a mutated LL-37 peptide or fragment thereof, comprising at least 18 consecutive amino acids of the sequence: N′-R I x Q R I K x F x R x L x x R x K x-C′; wherein x is any amino acid residue; provided that at least one x, suitably at least two x, more suitably at least three x, and still more suitably four or more x are different to the corresponding residue of wild-type LL-37. Alternatively, one or more (e.g. 1 or 2) x may be a deletion.

Most suitably, the AMP of the invention comprises a sequence selected from the group consisting of: (i) LLGVFFRKRKAKIGKKFKRILQRIKDFRRNLVPRIKS (SEQ ID NO: 2); (ii) FKRILQRIKDFRRNLVPRIKS (SEQ ID NO: 3); (iii) VKRIVQRIKVFLRNLMPRSKS (SEQ ID NO: 4); (iv) FRRIVQRIKDFLRNLV MRRKN (SEQ ID NO: 5); and (v) FKRIVQRIKDFLRKLVPRIMS (SEQ ID NO: 6).

In other embodiments the AMP of the invention may comprise any of the sequences selected from SEQ ID NO: 7 to 21 (see e.g. Table 3).

An AMP of the invention may be amidated at the C-terminus, and/or may have one or more intramolecular non-peptide bond (e.g. a hydrocarbon staple). In some embodiments, the AMP or derivative of the invention is linked to, associated with or attached/conjugated to another (e.g. non-AMP) moiety. Accordingly, the methods of the invention further comprise conjugating the AMP to another moiety, particularly a non-AMP moiety. The other/non-AMP moiety can be a peptide, a nucleic acid or another compound, such as a therapeutic molecule. In some embodiments, the means of linkage, association, attachment or conjugation is readily cleavable by means of an enzymatic reaction or other chemical process/degradation.

According to the invention, the AMPs isolated by the invention are preferably non-naturally occurring peptide sequences that have antimicrobial activity. Advantageously, the AMPs of the invention have antimicrobial activity against more than one microbe, and more suitably against at least one bacterial species and at least one virus. The AMPs of the invention further possess some inherent protease resistance. Suitably, where appropriate, the protease resistance exhibited by the AMP is greater than the protease resistance exhibited by the sequence from which the AMP was derived, such as wild-type LL-37 or a fragment thereof.

In a further aspect of the invention there is provided a nucleic acid molecule comprising a nucleic acid sequence encoding an AMP of the invention, optionally further encoding a non-AMP peptide or moiety and optionally further comprising regulatory nucleic acid sequences. An expression vector comprising a nucleic acid molecule of the invention is also provided. Hence, the methods of the invention may further comprise isolating the nucleic acid construct encoding an AMP of the invention and inserting it into an expression vector or construct.

The invention further provides therapeutic compositions and molecules comprising an AMP of the invention. Therefore, the methods of the invention may further comprise formulating a pharmaceutical composition comprising an AMP. The composition of the invention may be formulated for topical application, for example, for use as a handwash. The handwash may be sporicidal or bacteriocidal. Preferred applications are against spores or bacterium selected from Bacillus anthracis and/or Clostridium difficile.

Optionally, the AMP may be a derivative or may be conjugated to or functionally linked to a non-AMP moiety, for example, a further therapeutic molecule (e.g. a therapeutic peptide or nucleic acid) or diagnostic agent. Non-limiting examples of non-AMP moieties and potential therapeutic molecules include nucleic acids (e.g. siRNA molecules), enzymes, hormones, cytokines, antibodies or antibody fragments, peptide fragments (e.g. peptides recognised by antibodies), analgesics, antipyretics, anti-inflammatory agents, antibiotics, antiviral agents, anti-fungal drugs, cardiovascular drugs, drugs that affect renal function and electrolyte metabolism, drugs that act on the central nervous system and chemotherapeutic drugs.

The invention also provides therapeutic compositions, such as a pharmaceutical composition comprising a nucleic acid construct encoding an AMP.

In another aspect, the invention provides an AMP or pharmaceutical composition of the invention for use in medicine. Accordingly, the invention also provides for the use of an AMP in the manufacture of a medicament for treating a medical condition or disorder in need thereof. For example, the use or the medical condition may be for the treatment of a microbial infection in a subject. Typically, the microbial infection is selected from bacteria, fungi, parasites and enveloped viruses. The bacterial infection requiring treatment may be selected from the group consisting of Bacillus anthracis, Burkholderia cepacia, Clostridium difficile, Staphlococcus aureus, Mycobacterium tuberculosis, Escherichia coli, Salmonella enterica, Pseudomonas aeruginosa and Francisella tularensis. Preferably, the infection is selected from Bacillus anthracis, Clostridium difficile Staphylococcus aureus, Streptococcus mutans, methicillin resistant Staphylococcus aureus, Pseudomonas aeruginosa and Escherichia coli. Most preferably, the bacterial infection is Bacillus anthracis. In the alternative, the infection may be Candida albicans. The medical treatment and, thus, the AMP or pharmaceutical of the invention may be suitable for the treatment of bacterial cells or spores, yeast or viral infection, and/or infected/transformed cells, such as cells infected by a parasite or virus, or a tumour cell. In another aspect, the invention acts as a sporicidal agent that can be used to kill bacterial spores which are produced by organisms, such as Bacillus anthracis, and survive in the environment, such as in soil. In another embodiment, the AMP of the invention is for use in treating a cancer, tumour or neoplasm in a subject. The subject may be an animal, such as a non-human mammal or a human. Suitably the subject is a human.

Specific AMPs and therapeutic molecules of the invention include linear or cyclic peptides, and combinations thereof, optionally modified at the N-terminus or C-terminus or both, as well as their salts and derivatives, functional analogues, retro-inverso or D-amino acids enantiomers thereof, and extended peptide chains carrying amino acids or polypeptides at the termini of the sequences.

The pharmaceutical composition may also be formulated as a liposome composition, for example, for use in delivering therapeutic peptides, nucleic acids or conjugated molecules of the invention to target cells or organs in vivo or ex vivo.

According to the invention, in vitro peptide display libraries are generated by any suitable means known to the person of skill in the art. For example, libraries of in vitro generated peptide-nucleic acid complexes may be suitably generated by an appropriate method such as described by Roberts, & Szostak, (1997, Proc. Natl. Acad. Sci. USA, 94, 12297-12302), Mattheakis et al., (1994, Proc. Natl. Acad. Sci. USA, 91, 9022-9026), Odegrip et al., (2004, Proc. Natl. Acad. Sci. USA, 101 2806-2810) and by WO2004/022746. In certain cases, such as where the maximum library size is within the limits of phage display technology or chemical synthesis, these in vivo methods may alternatively be used. The libraries of peptide-nucleic acid complexes (preferably in vitro generated) are then selected according to their ability to resist protease degradation and to target a selected membrane/membrane composition of interest.

The invention further comprises AMP libraries, as used in the methods of the present invention. The AMP libraries of the invention are composed of, for example, peptides or peptide derivatives such as peptide mimetics and peptide analogues composed of naturally occurring or non-natural amino acids.

In one step of the method of the invention, library members encoding AMPs are selected by removing peptide-nucleic acid complexes encoding non-protease resistant peptide complexes from the mixture. Conveniently, a predetermined quantity (and/or activity) of one or more selected protease is added to the mixture of peptide-nucleic acid complexes so that susceptible peptides are degraded (by proteolysis). Degraded peptides cause dissociation of their respective peptide-nucleic acid complex, such that the nucleic acid encoding the susceptible peptide is removed from the selection process. It is convenient to use more than one protease so that the selected AMPs have at least a level of resistance to more than one protease. Suitably, the proteases are selected from the group consisting of animal (e.g. human), bacterial, fungal, parasitic and viral proteases. More suitably, the proteases are selected from animal/human, bacteria, yeast and/or parasite proteases.

In another step of the method of the invention, library members encoding AMPs are further selected by exposing them to membrane compositions and removing peptide-nucleic acid complexes that do not associate with the membranes. Hence, those peptides that do not associate with the target membrane and, accordingly, the corresponding nucleic acids that encode non-membrane-associating peptides can be removed. The non-associating complexes can be removed by any convenient means, such as centrifugation; optionally including one or more subsequent washing steps. Alternatively (or additionally) the mixture of membranes and peptide-nucleic acid complexes can be exposed to one or more protease to remove non-associating and/or susceptible peptides. In this way, AMPs capable of associating with the target membrane of a cell or vesicle may be recovered and characterised. By “associating with”, it is meant that the peptide forms a physical interaction with the membrane or components of the membrane (such as lipids, proteins and/or carbohydrate moieties), which may include residing on the membrane (carpet model), inserting into, or (in part) translocating across the membrane (known as barrelstave, wormhole or toroidal, and aggregate channel models)—as reviewed by Giuliani et al., (2007, “Antimicrobial peptides: an overview of a promising class of therapeutics” CEJB 2(1), 1-33).

The invention also provides for the selection of a peptide-nucleic acid complex encoding an AMP linked to one or more AMPs or any other combinations that can be envisaged by one skilled in the art. For example, in such embodiments one or more (preferably each) of the members of the library of nucleic acid sequences may encode 2, 3, 4 or more AMPs or potential AMP sequences.

The invention further provides for the selection of a peptide-nucleic acid complex encoding an AMP linked to one or more non-antimicrobial moieties. A suitably non-antimicrobial moiety may be a protease resistance-conferring moiety, such as a protease-resistant/peptide-stabiliser sequence, for example, as described in our co-pending patent application WO2006/097748.

### BRIEF DESCRIPTION OF THE DRAWINGS

The invention is further illustrated by the accompanying drawings in which:

FIG. 1 shows the results of a membrane-binding ELISA assay for a number of AMPs, following pre-exposure to either a subtilisin or a subtilisin/V8 protease cocktail. The resistance of each AMPs to proteolytic degradation by the proteases is demonstrated by the preservation of the ELISA signal after treatment with protease: (i) 0.1 μml subtilisin—grey box; (ii) 0.1 U/ml subtilisin and 0.1 μml V8 protease black box); (iii) blank control—light box.

FIG. 2 depicts the results of antimicrobial assays with selected AMPs derived from an LL-37 peptide against Bacillus anthracis spores over a 24 hour period, in comparison to: wild-type LL-37 (solid line and cross); MA-39 (LL-37 with an additional methionine and alanine on the N-terminus; solid line and solid circle); and MA-22 (KR-20 with an additional methionine and alanine on the N-terminus; solid line and open square). Results are depicted for selected AMPs: C11 (solid line and solid triangle); G11 (a dotted line and cross); F11 (solid line and open triangle); and H04 (solid line and open circle). A negative control without AMP peptide is denoted by a dashed line with a solid square.

FIG. 3 depicts the results of antimicrobial assays with selected AMPs derived from an LL-37 peptide against Bacillus anthracis in vegetative state over a 24 hour period, in comparison to: wild-type LL-37 (solid line and cross), MA-39 (LL-37 with an additional methionine and alanine on the N-terminus; solid line and solid circle) and MA-22 (KR-20 with an additional methionine and alanine on the N-terminus; solid line and open square). Results are depicted for selected AMPs: C11 (solid line and solid triangle); G11 (a dotted line and cross); F11 (solid line and open triangle); and H04 (solid line and open circle). A negative control without AMP peptide is denoted by a dashed line with a solid square.

FIG. 4 depicts the results of antimicrobial assays with selected AMPs derived from wild-type LL-37 peptide against Burkholdena cepacia in vegetative state over a 24 hour period, in comparison to wild-type LL-37 (solid line and cross). Results are depicted for selected AMPs: C11 (solid line and solid triangle); G11 (a dotted line and cross); F11 (solid line and open triangle); and H04 (solid line and open circle). A negative control without AMP peptide is denoted by a solid line with a solid square.

FIG. 5 depicts the results of antimicrobial assays with serial dilutions of selected AMPs, derived from wild-type LL-37 peptide, against Staphylococcus aureus, in comparison to wild-type MAF-23 (solid line and solid square) and MAF-23 acetylated at the N-terminus (solid line and open diamond). Results are depicted for selected AMPs: F11 (solid line and open triangle); N-terminally acetylated F11 (dashed line and solid triangle); and H04 N-terminally acetylated (dashed line and solid circles). A negative control with an irrelevant peptide is denoted by a dashed line with an open square.

FIG. 6 depicts the results of antimicrobial assays with serial dilutions of selected AMPs, derived from wild-type LL-37 peptide, against Candida albicans, in comparison to wild-type MAF-23 (solid line and solid square) and MAF-23 acetylated at the N-terminus (solid line and open diamond). Results are depicted for selected AMPs: F11 (solid line and open triangle); N-terminal acetylated F11 (dashed line and solid triangle); and H04 N-terminally acetylated (dashed line and solid circles). A negative control with an irrelevant peptide is denoted by a dashed line with an open square.

FIG. 7 shows the anti-viral activity of selected AMPs derived from wild-type LL-37 against Vaccinia virus, in comparison to wild-type LL-37: PBS buffer (negative control; columns A and E); wild-type LL-37 (columns B and F); AMP F11 (columns C and G); AMP H04 (columns D and H). Columns A to D—assays include additional treatment with an intracellular mature virus neutralising serum (RIMV).

### DETAILED DESCRIPTION OF THE INVENTION

In order to assist with the understanding of the invention several terms are defined herein. All references cited herein are incorporated by reference in their entirety. Unless otherwise defined, all technical and scientific terms used herein have the same meaning as commonly understood by one of ordinary skill in the art to which this invention belongs.

The terms “peptide”, “antimicrobial peptide” or “AMP” as used herein refer to a plurality of amino acids joined together in a linear or circular chain, preferably linear. The term oligopeptide is typically used to describe peptides having between 2 and about 50 or more amino acids. Peptides larger than about 50 are often referred to as polypeptides or proteins. For purposes of the present invention, the terms “peptide”, and “antimicrobial peptide” or “AMP” are not limited to any particular number of amino acids. Preferably, however, they contain about 6 to about 100 amino acids, or about 12 to about 50 residues, such as between about 15 and about 45 residues, suitably between about 18 and about 40 residues and more suitably between about 20 and about 37 residues. In another embodiment the AMP may conveniently contain about 20 to about 24 amino acid residues. For example, an AMP identified according to the methods of the invention may be 18, 19, 20, 21, 22, 23 or 24 amino acids in length. It should be understood that an isolated AMP of the invention may comprise or equally it may consist of the number of amino acids indicated herein.

An AMP of the invention may be a mutated fragment of a wild-type AMP, such as LL-37. Thus, the AMP is conveniently derived from a wild-type protein or peptide sequence. The AMP may comprise an addition peptide sequence or sequences at the N- or C-terminus of the corresponding wild-type peptide sequence from which it is derived, e.g. the dipeptide sequence met-ala may be included at the N-terminus. In this case, the full length of an AMP may be two amino acids longer than indicated above.

“Antimicrobial peptides” (AMPs) as used herein are amino acid sequences (as described above), which may contain naturally as well as non-naturally occurring amino acid residues. Therefore, so-called “peptide mimetics” and “peptide analogues”, which may include non-amino acid chemical structures that mimic the structure of a particular amino acid or peptide, may also be “antimicrobial peptides” within the context of the invention. Such mimetics or analogues are characterised generally as exhibiting similar physical characteristics such as size, charge or hydrophobicity, and the appropriate spatial orientation that is found in their natural peptide counterparts. A specific example of a peptide mimetic compound is a compound in which the amide bond between one or more of the amino acids is replaced by, for example, a carbon-carbon bond or other non-amide bond, as is well known in the art (see, for example Sawyer, in Peptide Based Drug Design, pp. 378-422, ACS, Washington D.C. 1995).

The present invention is directed towards the identification and characterisation of AMPs from amongst a population (or library) of peptides—i.e. potential or putative AMPs that may be expressed from a library of nucleic acid sequences. Although the term “peptide” is used herein, it will be understood that the present invention does not preclude identification of AMPs or larger peptide domains and motifs that would perhaps under conventional nomenclature be appropriately referred to as polypeptides or proteins.

Furthermore, the term “antimicrobial peptide” (AMP) may include peptides that associate with or insert into a membrane or lipid bilayer. In some cases, the AMP may even traverse a membrane so that the AMP and any associated non-AMP moieties pass from one side of the membrane to the other. AMPs that insert into a membrane may be considered to span the membrane. By “span” it is meant that an AMP may fully insert (penetrate) into the membrane, or it may simply insert a few amino acids into the membrane. Thus, when an AMP spans a membrane, at least a portion of the AMP remains within the membrane. In some beneficial embodiments, an AMP according to the invention fully spans or penetrates a target membrane. Typically, a membrane-spanning domain of a protein is in the region of 20 to 25 amino acids in length and, therefore, where an AMP associates with a target membrane by insertion it may conveniently include a membrane-spanning region.

Typically, the AMP is derived from the LL-37 peptide, i.e. N′-LLGDFFRKSKEKIGKEFKRIVQRIKDFLRNLVPRTES-C′ (SEQ ID NO: 1), which is a peptide fragment of the human cathelicidin protein. The AMP may thus be selected from a library of mutant LL-37 peptides (or fragments thereof). A selected AMP of the invention contains 1 or more, suitably 3 or more mutations relative to the wild-type peptide sequence from which it is derived. A preferred form of mutation is an amino acid substitution. Alternatively, the AMP may be derived from other cathelicidins, such as from mammals, fish or reptiles; e.g. as described by Lehrer & Ganz (2002), “Cathelicidins: a family of endogenous antimicrobial peptides”, Curr. Opin. Hematol., 9(1), 18-22; Wang et al., (2008), “Snake cathelicidin from Bungarus fasciatus is a potent peptide antibiotics”, PLoS One, 16, 3(9), e3217; and Zanetti (2004), “Cathelicidins, multifunctional peptides of the innate immunity”, J. Leukocyte Biology, 75, 39-48. In particular, peptide fragments from Rhesus LL-37, Monkey RL-37, Rabbit CAP18, Mouse CRAMP, rat rCRAMP, Guinea pig CAP11, Chicken Fowlicidins 1-3, Rainbow trout rtCATH-1 & rtCATH-2, atlantic salmon asCATH-1 & asCATH-2, hagfish HFIAP-1 and HFIAP-2 may also be used as a starting point for selection of AMPs of the invention. Alternatively, the AMP of the invention may be derived from a peptide or peptide fragment of a defensin, as described in the defensin knowledgebase http://defensins.bii.astar.edu.sg/; or those mentioned in the antimicrobial peptide database: Wang & Wang, (2004), “APD: the Antimicrobial Peptide Database”, Nucleic Acids Research, 32, D590-D592; http://aps.unmc.edu/AP/main.php.

By “derived from” it is meant that the peptide concerned includes one or more mutations in comparison to the primary amino acid sequence on which is was based. Thus an AMP of the invention may derived from a wild-type protein/peptide sequence, such as from LL-37. Similarly, by the term “derivative” of an AMP it is meant a peptide sequence that has antimicrobial activity, that has some resistance to proteolytic degradation (and optionally also includes associated/conjugated non-AMP moieties), but that further includes one or more mutations or modifications to the primary peptide sequence of an AMP first identified by the methods of the invention. Thus, a derivative of an AMP may have one or more, e.g. 1, 2, 3, 4, 5 or more chemically modified amino acid side chains, which have been introduced into an AMP. In addition or in the alternative, a derivative of an AMP may contain one or more, e.g. 1, 2, 3, 4, 5 or more amino acid mutations, substitutions or deletions to the primary sequence of an identified AMP. Thus, the invention encompasses the results of maturation experiments conducted on an AMP to improve one or more characteristics of the AMP. For instance, 1, 2, 3, 4, 5 or more amino acid residues of an AMP sequence may be randomly or specifically mutated (or substituted) using procedures known in the art (e.g. by modifying the encoding DNA or RNA sequence), and the resultant library/population of derivatised peptides may be selected—by any known method in the art—according to predetermined requirements: such as improved antimicrobial activity against a particular microbe (or type of microbe, e.g. bacteria); improved resistance to particular protease(s); improved specificity against particular target microbes; or improved drug properties (e.g. solubility, bioavailability, immunogencity etc.). Peptides selected to exhibit such additional or improved characteristics and that display antimicrobial activity may be considered to be derivatives of AMPs and fall within the scope of the invention.

By “antimicrobial” it is meant a peptide that kills, inhibits or otherwise prevents the multiplication of a pathogenic organism. The pathogenic organism includes microbes, such as bacteria, fungi, viruses and parasites. An AMP of the invention beneficially has antimicrobial activity against more than one microbe and, preferably has antimicrobial activity against more than one type of organism (e.g. bacteria and virus; bacteria and parasite; bacterial and fungus etc.). An AMP of the invention may have antimicrobial activity against bacterial spores and/or bacterial cells (e.g. bacteria in the vegetative state). Preferred microbes are indicated elsewhere herein.

The term “membrane” includes the membranes of any artificial or naturally occurring membrane that comprises a monolayer or bilayer of aliphatic molecules, such as fatty acid or lipid molecules. Thus, the term includes the membranes of micelles, liposomes, or other vesicles known to the person of skill in the art, and any type of naturally occurring cell, including bacterial, fungus, plant, animal or human (including, for example, blood cells, epithelial cells, including skin cells and gut wall cells). Preferably, the membrane is a lipid bilayer and more preferably the membrane is derived from a bacterium.

A “non-AMP moiety” as used herein, refers to an entity that cannot by itself provide sufficient antimicrobial activity or resistance to cause a measurable desired and significant antimicrobial effect relative to placebo. Such non-AMP moieties include nucleic acids and other polymers, peptides, proteins, peptide nucleic acids (PNAs), antibodies, antibody fragments, and small molecules, amongst others. Advantageously, a non-AMP moiety may be a therapeutic molecule.

The term “amino acid” in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as 8-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The present invention is directed towards the identification and characterisation of AMPs from amongst a population (or library) of peptides—i.e. potential or putative AMPs. In particular, the AMPs of the invention are selected using in vitro display of in vitro generated libraries of peptides.

The terms “in vitro display”, “in vitro peptide display” and “in vitro generated libraries” as used herein refer to systems in which peptide libraries are expressed in such a way that the expressed peptides associate with the specific nucleic acids that encoded them, and in which such association does not follow or require the transformation of cells or bacteria with the said nucleic acids. Accordingly, these systems can be considered to be “acellular”. Such systems contrast with phage display and other “cellular” or “in vivo display” systems in which the association of peptides with their encoded nucleic acids follows the transformation of cells or bacteria with the nucleic acids.

In some cases it may be desirable to conjugate an antimicrobial peptide of the invention to a non-AMP moiety. The term “conjugate” is used in its broadest sense to encompass all methods of attachment or joining that are known in the art. For example, the non-AMP moiety can be an amino acid extension of the C- or N-terminus of the AMP. In addition, a short amino acid linker sequence may lie between the AMP and the non-translocating moiety. The invention further provides for molecules where the AMP will be linked, e.g. by chemical conjugation to the non-AMP moiety optionally via a linker sequence. Typically, the AMP will be linked to the other moiety via sites that do not interfere with the activity of either moiety.

The term “conjugated” is used interchangeably with the terms “linked”, “bound” “associated” or “attached”. A wide range of covalent and non-covalent forms of conjugation are known to the person of skill in the art, and fall within the scope of the invention. For example, disulphide bonds, chemical linkages and peptide chains are all forms of covalent linkages. Where a non-covalent means of conjugation is preferred, the means of attachment may be, for example, a biotin-(strept)avidin link or the like. Antibody (or antibody fragment)-antigen interactions may also be suitably employed to conjugate an AMP of the invention to a non-AMP moiety. One suitable antibody-antigen pairing is the fluorescein-antifluorescein interaction.

### Selection/Peptide Engineering Procedures

To date, novel peptides have been engineered through the use of two different approaches. The first approach produces candidate peptides by chemically synthesising a randomised library of 6-10 amino acid peptides (J. Eichler et al., 1995, Med. Res. Rev. 15:481-496; K. Lam, 1996, Anticancer Drug Des. 12:145-167; M. Lebl et al., 1997, Methods Enzymol. 289:336-392). In the second approach, candidate peptides are synthesised by cloning a randomised oligonucleotide library into an Ff filamentous phage gene, which allows peptides that are much larger in size to be expressed on the surface of the bacteriophage (H. Lowman, 1997, Ann. Rev. Biophys. Biomol. Struct. 26:401-424; G. Smith et al., 1993, Meth. Enz. 217:228-257). Randomised peptide libraries up to 38 amino acids in length have also been made, and longer peptides are achievable using this system. The peptide libraries that are produced using either of these strategies are then typically mixed with a pre-selected matrix-bound protein target. Peptides that bind are eluted, and their sequences are determined. From this information new peptides are synthesised and their biological properties are determined. Phage display has previously been used to identify antimicrobial peptides, see for example, Tanaka et al., (2008), “Novel Method for Selection of Antimicrobial Peptides from a Phage Display Library by Use of Bacterial Magnetic Particles”, Applied and Environmental Microbiol., 74, 7600-7606; and Pini et al., (2005), “Antimicrobial Activity of Novel Dendrimeric Peptides Obtained by Phage Display Selection and Rational Modification”, Antimicrob. Agents Chemother. 49, 2665-2672.

A particular disadvantage of such prior art procedures is that the size of the libraries that can be generated with both phage display and chemical synthesis is limited to within the 10⁶-10⁹ range. This limitation has resulted in the isolation of peptides of relatively low affinity, unless a time-consuming maturation process is subsequently used. This library-size limitation has led to the development of techniques for the in vitro generation of peptide libraries including mRNA display (Roberts, & Szostak, 1997, Proc. Natl. Acad. Sci. USA, 94, 12297-12302), ribosome display (Mattheakis et al., 1994, Proc. Natl. Acad. Sci. USA, 91, 9022-9026) and CIS display (Odegrip et al., 2004, Proc. Natl. Acad. Sci. USA, 101 2806-2810) amongst others. These libraries are superior to phage display libraries (and other in vivo-based procedures), in that the size of libraries generated by such methods is 2-3 orders of magnitude larger than is possible with phage display. This is because unlike techniques such as phage display, there are no intermediate in vivo steps.

The present invention represents a significant advance in the art of peptide drug development by allowing screening of in vitro generated libraries for antimicrobial properties. In vitro generated nucleic acid libraries encoding a plurality of peptides are synthesised and initially selected for their ability to resist protease degradation. When a library member (peptide) is cleaved by the proteases used in this stage, the peptide-nucleic acid complex dissociates so that—at the end of the relevant round of the selection procedure—it is not possible to recover the nucleic acid that encoded that peptide as being associated with a peptide bound to a target membrane. The proteolytic conditions can be selected according to requirements. For example, the type of enzyme or protease, number of proteases, concentration, and activity of the proteases may be chosen on a case by case basis. Typically, the protease enzymes used are those that are appropriate for the intended use of the AMP. Thus, where it is desired that the AMP will have antimicrobial activity against a particular target bacterial, viral or fungal type, the protease(s) selected may be those that are endogenous to the target. Furthermore, if it is desired that the AMP is to have a therapeutic use in an animal, such as a human, the protease(s) selected may be those that are endogenous to the target animal (e.g. those in the circulating blood flow of the animal concerned). Furthermore, the selection of proteases and protease conditions may be dependent on the choice of target membrane composition so as not to adversely affect the integrity of the target membrane. By way of example, the proteases are typically selected from one or more of chymotrypsin, trypsin, aminopeptidases, elastases, thermolysin, subtilisin, V8 protease and Entamoeba histolytica cysteine protease, but any other appropriate protease can also be used.

The proteases may be used alone or in any combination and, furthermore, when more than one round of selection is used, the protease(s) used may be different in one or more rounds. Conveniently, in a last round of selection all appropriate/desirable proteases are advantageously included to increase the likelihood that the selected peptides have a good level of protease resistance. The amount of each protease used can vary according to the protease concerned. For example, the enzymes might be used in the concentration range of about 0.001 to about 10 units/ml, such as about 0.01 to about 1 units/ml, or about 0.05 to about 0.5 units/ml. Suitably, the concentration of protease may be between about 0.075 and about 0.25 units/ml. In some embodiments the concentration of each protease used is about 0.1 units/ml. The peptide-nucleic acid complexes may be incubated with the one or more proteases for any suitable time period. For example, up to about 2 hours; from about 1 minute to about 1 hour; or from about 10 minutes to about 45 minutes. In some embodiments an incubation time of about 30 minutes is used. For convenience, the protease(s) may be conjugated to a solid support so that they can be readily removed from the mixture. A suitable solid support is a bead, such as a magnetic bead, but may be a tube or plate. In one suitable example the bead is an agarose bead that can be separated from the mixture under gravity, for example, using centrifugation. Suitable centrifugation conditions are known. In one example, the mixture is centrifuged at 16,000 g for 1 minute.

Library members are also selected based on their ability to associate with a target membrane. Conveniently, this step is carried out subsequent to exposure of the AMPs to the one or more proteases; although it may be carried out before. Library members that survived the proteolysis reaction, but which are incapable of associating with a target membrane (and free nucleic acid molecules from dissociated complexes) can be removed from the selection, for example, by washing or other appropriate methods known to those skilled in the art. It is worth noting that a particular limitation of phage display libraries over the methods of the present invention is the inherent non-specific binding by phage particles to cell membranes, such non-specific binding being well known to those skilled in the art, which could significantly compromise the ability to select for specific membrane-associating peptides from those non-specific binding complexes.

A desired membrane composition may be prepared as follows. By way of example, bacterial membranes may be prepared by the method of Horstman and Kuehn (J.B.C. Vol. 275, No. 17, pp. 12489-12496, 2000). An overnight culture (e.g. 250 ml) of bacterial, such as Bacillus subtilis (NCIMB 3610), is harvested (e.g. at 6000 g, 10 min) and resuspended in (10 mM) HEPES (pH 7.8), 0.5 mM EDTA (HE buffer). Cells are lysed, for example, using a French Pressure cell at 20,000 psi, and intact cells are removed, conveniently by centrifugation (e.g. 6000 g, 10 min). The supernatant containing membranes may be applied to a sucrose cushion (e.g. 2 ml 55% sucrose in HE, 0.5 ml 5% sucrose in HE) and centrifuged (e.g. at 150,000 g for 3 hr). Membranes can then be removed from the interface via a 19-gauge needle and syringe. Membranes can be diluted (e.g. five fold in 10 mM HEPES (pH 7.8)), prior to use. A similar procedure may be used for preparing the membranes of other bacterial species. Any other known means of harvesting and preparing membrane compositions known to the person of skill in the art may also be used.

Conveniently, to aid in the separation of membrane associated complexes from free complexes, the membranes may be associated with or otherwise attached to a solid support. By way of example, the solid support may be the surface of a plate, tube or well; alternatively the solid support may be a bead, such as a magnetic or agarose bead. In one example, the bead is a polystyrene-coated magnetic bead. The solid support may be coated with membrane using any appropriate method. For instance, a membrane composition, such as Bacillus subtilis membranes, are added to magnetic beads, for example, polystyrene-coated magnetic beads (PM-30-10, Spherotech, Lake Forest, Ill., USA), in suitable buffer (such as PBS) and incubated for a period of time. The incubation can conveniently be carried out at room temperature whilst mixing on a rotary mixer. Before use the beads may be washed, for example, 3 times with PBS buffer. After a suitable incubation time, magnetic beads may be pelleted under gravity and/or magnetic force, for example, so as to separate membrane-bound peptide-nucleic acid complexes from non-associated complexes in solution.

In the alternative, for example, when the membranes are not associated with a solid support, the mixture of membranes and peptide-nucleic acid complexes may first be centrifuged to pellet the membranes and associated complexes, leaving unassociated complexes in the supernatant.

In the above way, the membrane-associated peptide-nucleic acid complexes can be readily separated (as a pellet) from non-associated complexes, which remain in free solution/suspension. Non-associated complexes may be removed by aspiration and, typically, with one or more washing steps using suitable buffers and/or detergents. A convenient buffer is PBS, but other suitable buffers known in the art may also be used.

The membrane-associated AMPs may then be recovered and individually characterised by sequencing the associated nucleic acid, and for example, expressing or synthesising the encoded AMP to confirm the desired membrane-associating and protease resistance properties.

Advantageously, the AMPs of the invention are isolated and individually characterised. However, a mixed population of AMPs may be obtained by the methods of the invention, e.g. where more than one peptide-nucleic acid complex associates with a membrane during the methods of the invention. In this event, the invention also encompasses a mixed population of AMPs.

The type of membrane used in the selection method can be selected on the basis of the type of antimicrobial activity that it is desired to achieve. Conveniently, the membranes of the microbe to be targeted by the AMPs of the invention will be used for selecting AMPs. However, the membranes of one organism of a particular genus may serve as a suitable substrate for the selection of AMPs having antimicrobial activity against a different member of the same genus. Thus, it may be convenient to use Bacillus membranes (e.g. from Bacillus subtilis) when a microbial target is a Bacillus bacterium, such as Bacillus anthracis. In other cases it may be convenient or desirable to use a different type of membrane or lipid bilayer. For example, the membrane may either be selected from a gram-negative bacterium (to obtain an AMP against a gram-negative bacterium), or from a gram-positive bacterium (to obtain an AMP against a gram-positive bacterium). In some embodiments, the membrane may be a viral envelope or a yeast or parasite membrane.

Optionally, the invention can be applied to the isolation of cell-type specific AMPs; for example, to distinguish between microbe (e.g. bacterial and/or fungal cells) and animal cell membranes. It may also be useful to distinguish between different types of microbe membrane. In other cases, it may be desirable to have a bacterial species-specific AMP. However, it can be particularly useful to select for/obtain multiple antimicrobial activities in a single AMP. In this case, in vitro generated nucleic acid libraries encoding a plurality of peptides may be synthesised and selected for association to a target membrane composition of interest, such as a population of Bacillus cells or membranes, after an earlier incubation with a different non-target cell population in order to remove cross-reactive AMPs (i.e. those AMPs that associate with the non-target cell-type). Means of carrying out such methods will be known to those skilled in the art. Typically, library members incapable of associating with the target membrane composition of interest are removed by washing or other appropriate methods that will be apparent to those skilled in the art. Membrane-associated complexes are then removed from the cell surface by a suitable means known to one skilled in the art. As in the above-described methods of the invention, only library members encoding an AMP remain within the associated population. The membrane-associated AMPs may then be recovered and individually characterised by sequencing the associated nucleic acid, and/or expressing or synthesising the encoded AMP to confirm the desired properties.

### Therapeutic Compositions

An AMP of the invention may be incorporated into a pharmaceutical composition for use in treating an animal; preferably a human. The AMP of the invention (or derivative thereof) may be used to treat one or more microbial infections or conditions, for example, selected from a bacterial, viral, fungal or parasitic infection in a subject. They may also be used to treat microbial contamination, e.g. of a surface such as skin. Preferred bacterial infections include Clostridium difficile, Pseudomonas aeruginosa, Bacillus anthracis, Streptococcus mutans, Staphylococcus aureus, methicillin resistant Staphylococcus aureus and Escherichia coli. Preferred viral infections include influenza virus, smallpox virux. Preferred fungal infections include Candida albicans. Preferred parasitic infections include Plasmodium, Toxoplasma gondii, Trypanosoma brucei and Schistosoma mansoni. Additional bacterial infections include Burkholderia cepacia and Francisella tularensis. In one preferred example the infection is a Bacillus anthracis bacterial infection. The therapeutic composition may be active against bacterial cells and/or against bacterial spores.

Furthermore, the ability of the AMP of the invention to target selected membranes enables the AMP of the invention to be used to target mammalian membranes; in particular, those that have transformed into cancer cells or display an altered membrane phenotype, e.g. due to infection, such as by a virus or parasite. In this way, the AMPs, AMP derivatives and AMP-based therapeutic molecules (e.g. conjugated to a non-AMP moiety) may also be used as cancer, tumour or anti-neoplasm therapeutics.

One or more addition pharmaceutically acceptable carrier (such as diluents, adjuvants, excipients or vehicles) may also be used. Suitable pharmaceutical carriers are described in “Remington's Pharmaceutical Sciences” by E. W. Martin.

Pharmaceutical formulations and compositions of the invention are formulated to conform with regulatory standards and can be administered orally, intravenously, topically, or via other standard routes. The pharmaceutical compositions may be in the form of tablets, pills, lotions, gels, liquids, powders, suppositories, suspensions, liposomes, microparticles or other suitable formulations known in the art.

In accordance with the invention, the AMP may be manufactured into medicaments or may be formulated into pharmaceutical compositions. When administered to a subject, an agent (including antimicrobial agents of the invention), is suitably administered as a component of a composition that comprises a pharmaceutically acceptable vehicle. The molecules, compounds and compositions of the invention may be administered by any convenient route, for example, intradermal, intramuscular, intraperitoneal, intravenous, subcutaneous, intranasal, epidural, oral, sublingual, intranasal, intravaginal, transdermal, rectally, by inhalation, or topically to the skin. Administration can be systemic or local. Delivery systems that are known also include, for example, encapsulation in microgels, liposomes, microparticles, microcapsules, capsules, etc., and can be used to administer the compounds of the invention. Any other suitable delivery systems known in the art is also envisioned in use of the present invention.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the invention can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the AMPs of the invention are formulated in accordance with routine procedures as a pharmaceutical composition adapted for oral administration (more suitably for human beings). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. One skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the AMP (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the AMP or derivative either alone or as a mixture in different ratios.

Typically, compositions for intravenous administration comprise sterile isotonic aqueous buffer. Where necessary, the compositions may also include a solubilising agent.

Additives may be included to enhance cellular uptake of the AMP (or derivative) of the invention, such as the fatty acids oleic acid, linoleic acid and linolenic acid.

In one pharmaceutical composition, an AMP (and its associated non-AMP moiety, e.g. a therapeutic molecule) may be mixed with a population of liposomes (i.e. a lipid vesicle or other artificial membrane-encapsulated compartment), to create a therapeutic population of liposomes that contain the AMP and optionally the non-AMP/therapeutic molecule. The therapeutic population of liposomes can then be administered to a patient by e.g. intra-venous injection. Where it is necessary for the therapeutic liposome composition to target specifically a particular cell-type, such as a particular bacterial, viral or fungal species, the liposome composition may additionally be formulated with an appropriate antibody domain or the like (e.g. Fab, F(ab)₂, scFv etc.) or alternative targeting moiety, which recognises the target cell-type. Such methods are known to the person of skill in the art.

The AMPs of the invention way also be formulated into compositions for topical application to the skin of a subject, such as a hand or body wash. Such compositions may be useful for use in domestic environments (in the home), as well as in commercial environments (e.g. hotels and restaurants), and particularly in medical/healthcare facilities such as hospitals. Hand/body washes comprising AMPs of the invention may be suitable for treating bacterial, viral, fungal and/or parasitic contamination. For treating bacterial contamination, the AMPs may be suitable for treatment of bacterial cells and/or spores. Preferred bacteria are Clostridium difficile, Bacillus anthracis, Pseudomonas aeruginosa, Streptococcus mutans, Staphylococcus aereus, methicillin resistant Staphylococcus aureus and Escherichia coli. Preferably the AMP is present in the hand or body wash at a concentration suitable to have a bacteriocidal or bacteriostatic effect on the target bacteria.

### Nucleic Acids and Peptides

The AMPs according to the invention and AMPs conjugated to non-AMP peptide moieties may be produced by recombinant DNA technology and standard protein expression and purification procedures. Thus, the invention further provides nucleic acid molecules that encode the AMPs, derivatives thereof, or therapeutic molecules according to the invention. For instance, the DNA encoding the relevant peptide can be inserted into a suitable expression vector (e.g. pGEM®, Promega Corp., USA), and transformed into a suitable host cell for protein expression according to conventional techniques (Sambrook J. et al., Molecular Cloning: a Laboratory Manual, Cold Spring Harbor Press, Cold Spring Harbor, N.Y.). Suitable host cells are those that can be grown in culture and are amenable to transformation with exogenous DNA, including bacteria, fungal cells and cells of higher eukaryotic origin, preferably mammalian cells. The AMPs may, for example, be grown in fusion with another protein and purified as insoluble inclusion bodies from bacterial cells. This is particularly convenient when the AMP to be synthesised may be toxic to the host cell. Alternatively, AMPs may be synthesised in vitro using a suitable in vitro (transcription and) translation system (e.g. the E. coli S30 extract system, Promega corp., USA).

The term “operably linked”, when applied to DNA sequences, for example in an expression vector or construct indicates that the sequences are arranged so that they function cooperatively in order to achieve their intended purposes, i.e. a promoter sequence allows for initiation of transcription that proceeds through a linked coding sequence as far as the termination sequence.

Having selected and isolated an AMP, an additional functional group such as a second therapeutic molecule may then be attached to the AMP by any suitable means. As discussed hereinbefore, an AMP may be conjugated to any suitable form of therapeutic molecule, such has an antibody, enzyme or small chemical compound. A preferred form of therapeutic molecule is an siRNA molecule capable of inducing RNAi in a target cell. Typically a chemical linker will be used to link an siRNA molecule to a peptide, such as an AMP. For example, the nucleic acid or PNA can be linked to the peptide through a maleimide-thiol linkage, with the maleimide group being on the peptide and the thiol on the nucleic acid, or a disulphide link with a free cysteine group on the peptide and a thiol group on the nucleic acid. The AMPs may also be conjugated to a molecule that recruits immune cells of the host. Such conjugated AMP molecules may be particularly useful for use as cancer therapeutics.

In a further alternative, the AMP may be directly conjugated to an antibody molecule, an antibody fragment (e.g. Fab, F(ab)₂, scFv etc.) or other suitable targeting agent, so that the AMP and any additional conjugated moieties are targeted to the specific cell population required for the treatment or diagnosis—for example, a particularly bacterial species or cancer cell.

The invention will now be further illustrated by way of the following non-limiting examples.

### EXAMPLES

Unless otherwise indicated, commercially available reagents and standard techniques in molecular biological and biochemistry were used.

### Materials and Methods

The following procedures used by the Applicant are described in Sambrook, J. et al., 1989 supra.: analysis of restriction enzyme digestion products on agarose gels and preparation of phosphate buffered saline.

General purpose reagents were purchased from SIGMA-Aldrich Ltd (Poole, Dorset, U.K.). Oligonucleotides were obtained from Eurogentec Ltd (Southampton, U.K.). Amino acids, and S30 extracts were obtained from Promega Ltd (Southampton, Hampshire, U.K.). Vent and Taq DNA polymerases were obtained from New England Biolabs (Cambridgeshire, U.K.). FITC labelled peptides were obtained from Pepscan Systems (Lelystad, Netherlands).

### Example 1

### a. Library Construction

In vitro (Cis-display) library construction was carried out generally as described by Odegrip et al. (2004, Proc. Natl. Acad. Sci. USA, 101 2806-2810). All enzymes were purchased from New England Biolabs (NEB Ltd., Hitchin, UK). All PCRs contained 12.5 μmol of each primer, 1 unit of Phusion DNA polymerase, 250 μM dNTP (NEB Ltd., Hitchin, UK) and 1× polymerase buffer. PCRs were carried out on a Techne Techgene PCR machine (Fisher Scientific, Loughborough, UK) for one cycle of 2 min at 95° C., followed by 20 to 30 cycles at 95° C., 15 sec; 60° C., 30 sec; 72° C., 30 sec, followed by an extension of 5 min at 72° C.

Library templates were derived from a codon-optimised reverse translation of the wild-type LL-37 sequence (Uniprot accession number: P49913). Two library constructs were designed using either doped or randomised sequences.
1. Doped full-length LL-37 sequence: LL-37 was doped at a ratio of 15% per base in the coding sequence.
2. Doped truncated LL-37 sequence: the 17-37 sequence was doped at a ratio of 15% per base.

Oligonucleotide design was as described in Table 1 (see below). Oligonucleotides were supplied by Sigma Genosys Ltd. (Haverhill, UK) or by GeneLink Inc. (Hawthorn, N.Y. USA). Library primers were designed to alter the appropriate sequence of LL-37 or a truncated version of LL-37 so that each base had a 15% variance around the original sequence. The libraries were appended to repA-CIS-ori DNA template as described in Odegrip et al. (2004) via PCR, using library primers as 5′- and ORIrev as 3′ primers. Finally, a 5′ promoter was added to library constructs via PCR, using the 131mer and ORIrev primers. PCR products were purified using a Wizard PCR Cleanup kit using manufacturers procedures (Promega UK Ltd., Southampton, UK).

TABLE 1
Oligonucleotide design for the amplification and the three library
constructs used in LL-37 protease maturation selections.

LL-37 amino acid sequence:
LLGDFFRKSKEKIGKEFKRIVQRIKDFLRNLVPRTES
Library 1: LL-37 FL doped:
5′-GGAAACAGGATCTACCATGGCC-
87G87G558567777777858*6G658*6G566*6G677558*6G*66777*6G858677575C65858
67766G56777787G85866887G575885858688*66658-GGCAGCGGTTCTAGTCTAGC-
3′
Library 2: KR-20 truncated doped:
5′-GGAAACAGGATCTACCATGGCC-
77766G858677575C6585867766G56777787G85866887G575885858688*66658-
GGCAGCGGTTCTAGTCTAGC-3′
ORIrev:
5′-TGCATATCTGTCTGTCCACAGG-3′
131mer:
5′-CGGCGGTTAGAACGCGGCTACAATTAATACATAACCCCATCCCCCTGTT
GACAATTAATCATGGCTCGTATAATGTGTGGAATTGTGAGCGGATAACAATTTCACA
CAGG-3′
R1recfor:
5′-GAACGCGGCTACAATTAATACATAACC-3′
TAC6:
5′-CCCCATCCCCCTGTTGACAATTAATC-3′
TAC6-9:
5′-CGGCTCGTATAATGTGTGGAATTGTGAGC-3′
NOT1RECREV:
5′-TGGTGAAGATCAGTTGCGGCCGCTAG-3′
Key:
5 = 85% G
6 = 85% A
7 = 85% T
8 = 85% C
* = 85% A with no T
G = G only
C = C only
N = G, A, T, C
B = G, T, C

### b. Bacillus subtilis Membrane Preparation

Membranes were prepared based on the method described by Horstman and Kuehn (J.B.C. Vol. 275, No. 17, pp. 12489-12496, 2000). Briefly, a 250 ml overnight culture of Bacillus subtilis (NCIMB 3610) was harvested (6000 g, 10 min) and resuspended in 10 mM HEPES (pH 7.8), 0.5 mM EDTA (HE buffer). Cells were lysed via French Pressure cell at 20,000 psi and intact cells were removed via centrifugation (6000 g, 10 min). The supernatant was applied to a sucrose cushion (2 ml 55% sucrose in HE, 0.5 ml 5% sucrose in HE) and centrifuged (150,000 g for 3 hr). Membranes were then removed from the interface via a 19-gauge needle and syringe. Membranes were diluted five fold in 10 mM HEPES (pH 7.8).

### c. Selection Against Bacillus subtilis Membranes

Generally, in vitro transcription and translation were carried out as described by Odegrip et al. (2004, Proc. Natl. Acad. Sci. USA, 101, 2806-2810). 20 μg of each library DNA template (encoding approximately 10¹³ library members) was added to 200 μl in vitro transcription and translation (ITT) mixture using an S30 lysate system for linear templates (Promega UK Ltd., Southampton, UK) for up to 60 min at 30° C. and then diluted 5-fold in Selection Buffer containing 2% BSA (Sigma Aldrich Company Ltd., Gillingham, UK), 0.1 mg/ml herring sperm DNA (Promega UK Ltd., Southampton, UK), in PBS (Dulbecco A, Oxoid, Basingstoke, UK). Proteases chymotrypsin and trypsin coupled to agarose beads (Sigma Aldrich Company Ltd., Gillingham, UK) were added to a concentration of 0.1 units/ml and the mixture was incubated for 30 minutes at room temperature. The beads were then removed by centrifugation at 16,000 g for 1 min. During rounds 3 and 4 of selection, subtilisin and V8 proteases (Sigma Aldrich Company Ltd., Gillingham, UK) were also added to a concentration of 0.1 units/ml. Mixtures were then centrifuged at 16,000 g, 1 min and the pellets were discarded.

Selections were carried out on a Kingfisher 96 well magnetic separator (Fisher Scientific, Loughborough, U.K.). 25 μl of polystyrene-coated magnetic beads (PM-30-10, Spherotech, Lake Forest, Ill., USA), were coated with 25 μl Bacillus subtilis membranes made up to a volume of 1 ml with PBS and incubated at room temperature whilst mixing on a rotary mixer. The beads were washed 3 times with 1 ml PBS and then added to the ITT diluted in Selection Buffer. Binding was allowed to continue for 1 hr. Beads were then removed from the selection mixture and washed once in selection buffer, 10 minutes. Beads were removed and washed five times in PBS for 15 minutes each. Bound DNA was then eluted from the beads via incubation at 65° C. in 1×PCR buffer for 10 minutes. Recovery PCRs were carried out using nested forward primers, Rlrecfor (rounds 1 and 2), TAC6 (round 3) or TAC₆₋₉ (round 4) and reverse primer, NOT1 recrev (see Table 1). PCR products were purified and appended to repA-CIS-ori via restriction ligation as described in Odegrip et al. (2004). Restriction ligation products were amplified by PCR using nested forward primers R1recfor, TAC6 or TAC₆₋₉ and reverse primer ORIrev, thereby producing input DNA for the next round of selection. Selection conditions for round 2 were as above with 5 μg DNA, 100 μl ITT mixture, 25 μl beads coated with 25 μl membranes. For rounds 3 and 4 the conditions were as above but using 2.5 μg DNA, 50 μl ITT.

### d. Peptide Expression

Output DNA from the fourth round of selection against Bacillus membranes was cloned into an expression vector called pFLH which appends a C-terminal peptide tag comprising 3 FLAG (DYKDDDDK) epitopes in tandem, followed by a 10×His to library peptides. Output DNA was digested with 50 units of Nco1 and Not1 respectively in a 100 μl volume, containing 0.1 mg/ml acetylated BSA and 1×NEB Buffer 4 (NEB Ltd, Hitchin, UK). Digested products were purified via phenol:chloroform extraction and ethanol precipitation. Products were then inserted into pFLH (which had previously been digested with Nco1 and Not1), using Quick Ligase (NEB Ltd, Hitchin, UK) according to the manufacturer's instructions. Ligated products were then purified via phenol:chloroform extraction and ethanol precipitation. Products were transformed into BL21 (DE3) Escherichia coli (Merck Chemicals Ltd, Nottingham, UK) via electroporation. Transformed bacteria were then plated onto agar plates containing 2×TY and kanamycin (50 μg/ml). Plates were incubated at 37° C. overnight. Individual transformant colonies were grown up in 1 ml 2×TY plus kanamycin (50 μg/ml) in 2 ml 96 well plates (Greiner) to O.D. 600 nm=0.6 in an orbital shaking incubator at 300 rpm (37° C.). IPTG was then added to 1 mM and cultures were incubated for 1 hr (37° C.). Cultures were centrifuged for 10 mins at 3000 g (4° C.). Supernatants were discarded and pellets were lysed using 50 μl/well BugBuster Mastermix™ (Merck Chemicals Ltd, Nottingham, UK).

AMPs were synthesised by Alta Biosciences (University of Birmingham, Birmingham, UK) using methods previously described (Thwaite et al., 2006). Stock solutions were prepared by reconstituting each peptide in sterile phosphate-buffered saline (PBS), 0.02% acetic acid, 0.4% BSA (Sigma-Aldrich Company Ltd., Poole, UK) and sterilising with MINISART® 0.45 μm filters (Sartorius Ltd., Surrey, UK), then freezing at −20° C. at 1 mg/ml aliquots prior to use.

### e. Binding Analysis by ELISA

To determine binding to membranes, lysates were diluted 1:50 into 4% milk protein (Marvel, Premier Foods Ltd, UK) in PBS. Bacillus subtulis membranes were diluted 1:100 in 10 mM HEPES (pH7.8) and then coated onto MaxiSorp™ polystyrene plates (Nunc™ brand, Fisher Scientific, Loughborough, UK) plates at 4° C. overnight. Plates were washed once with PBS and then blocked for 1 hour with 4% milk protein in PBS. Plates were washed once with PBS and protease-treated solutions were added and incubated for 30 mins at room temperature. Plates were then washed four times with PBS. M2 anti-FLAG-HRP antibody—diluted 1:5000 in 4% milk protein in PBS—(Sigma Aldrich Company Ltd, Gillingham, UK), was added and incubated for 30 min at room temperature. Plates were then washed five times with PBS. The assay was developed with SureBlue TMB peroxidase substrate (Insight Biotechnology, Middlesex, UK) and read at 450 nm.

### f. Analysis of Protease Resistance

To determine proteolytic resistance, peptide lysate solutions at 1:25 in 4% milk protein in PBS were pre-incubated with either subtilisin or V8 protease (0.05 and 01 units/ml each) 30 minutes at room temperature. Proteolysis was then inhibited via addition of an equal volume of 4% milk protein in PBS containing Complete protease inhibitor cocktail—1 tablet dissolved in 20 ml of buffer—(Roche Diagnostics Ltd, Burgess Hill, UK). Binding, washing and detection steps were as described for the binding assay.

### g. Antibacterial Time-Kill Assays

Strains were grown to mid-exponential phase and aliquots of these cultures containing approximately 10⁵-10⁶ cfu/ml were separately exposed to PBS (control) or 102 μg/ml peptide (test). Spores of appropriate strains were also tested at the same concentration. Cultures were shaken at 180 rpm throughout the assay. Samples were taken at 0, 0.5, 1, 2, 3, 4 and 24 hours, then serially diluted in PBS and enumerated on agar. Viable cfu/ml counts were obtained following suitable incubation and were standardised to a percentage growth compared to that recorded at T=0 for each assay.

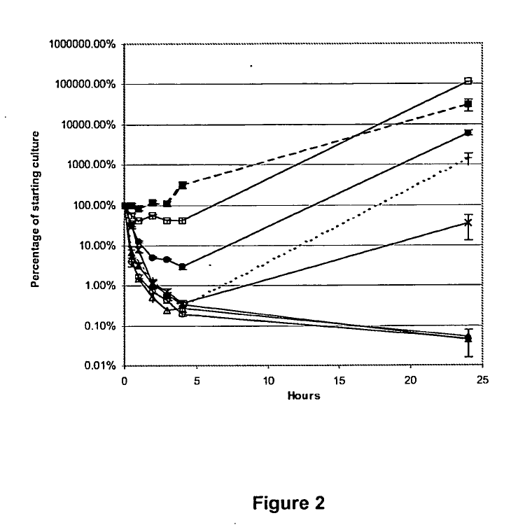

Peptides were synthesised alongside the three parent peptides (LL-37, MA-39, MA-22) and were tested on B. anthracis UM23-CI2 spores and vegetative cells. All AMPs caused between 99 and 99.9% reduction of the starting culture concentration of spores after 4 hours incubation, except for MA-39 (˜95% reduction), MA-22 (˜60% reduction) and A11 (no reduction at the concentration of peptide used) (see FIG. 2). After 24 hours, B. anthracis cultures exposed to C11, F11 and H04 were still further reduced to between 99.9 and 99.99% of the starting culture (see FIG. 2), while the bacterial cultures had fully revived where exposed to the wild-type peptides.

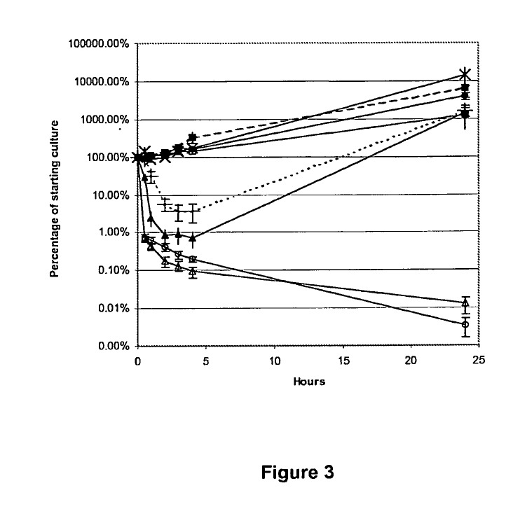

Against exponential phase cells each of C11, F11, G11 and H04 caused a significant reduction of the starting culture concentration over the 4 hour period: between 90 and 99% for G11, and between 99 and 99.9% for the others (see FIG. 3). The loss in activity was expected for the “wild-type” peptide sequences of LL-37, MA-39 and MA-22 as these are unaltered peptides and so still susceptible to proteolytic degradation over the time course. After 24 hours, the bacterial cultures exposed to F11 and H04 AMPs showed further reductions in cell number (see FIG. 3).

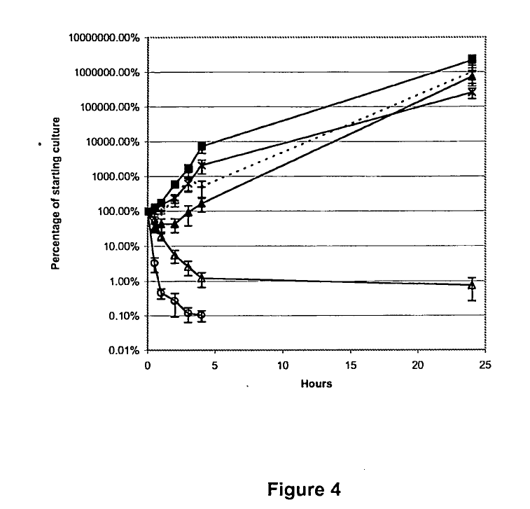

Following the B. anthracis assays, peptides were tested against a range of other category II organisms, including Burkholderia cepacia J2540, and showed good anti-bacterial properties. C11 and G11 were demonstrated to prevent B. cepacia from growing over 4 hours. Similarly, F11 caused an approximate 99% reduction of the starting culture of B. cepacia after 4 hours. H04 was also demonstrated to lower the B. cepacia bacterial count by 99.9% over the 4 hour time period. After 24 hours no bacteria were detectable in the assay containing H04 (see FIG. 4).

Significantly, native LL-37 had no effect on the growth of B. cepacia (as shown in FIG. 4), which demonstrates both the greater efficacy of AMPs of the invention and the potential for broader spectrum antimicrobial activity.

### h. In Vitro Assays for Antimicrobial Activity

The antimicrobial activity of some AMPs were determined as 50% and 90% Inhibitory Concentration (IC₅₀ and IC₉₀ respectively), i.e. the lowest peptide concentration at which bacterial growth was inhibited by 50% or 90% relative to a control containing no peptide. The ICs of each peptide were determined using the modified microtitre broth dilution method (Steinberg et al., 1997). Liquid bacterial cultures were grown to mid-exponential phase as described in Example 1 g and diluted to 1×10⁵ to 1×10⁶ cfu/ml. Spores of appropriate strains were also tested at the same concentration. Assays were performed as previously described (Thwaite et al., 2006) across concentration ranges of 62.5 ng/ml to 128 μg/ml

The activity of peptides against each of the bacterial strains described above was determined by measuring the inhibitory concentration values as previously described (see Table 2). Against B. anthracis spores peptides C11, F11 and H04 all possessed better IC50s that the wild type LL-37. In exponentially growing B. anthracis, Bacillus cepacia and Escherichia coli peptides F11 and H04 were notably improved.

TABLE 2
IC50 and IC90 for the peptides against B. anthracis spores and vegetative cells, B. cepacia and E. coli.

| | C11 | | F11 | | G11 | | H04 | | LL-37 | |
|---|---|---|---|---|---|---|---|---|---|---|
| Organism | IC50 | IC90 | IC50 | IC90 | IC50 | IC90 | IC50 | IC90 | IC50 | IC90 |
| B. anthracis | 31.3-62.5 | 62.5-125 | 31.3-62.5 | 31.3-62.5 | 125-250 | 125-250 | 15.6-31.3 | 15.6-31.3 | 62.5-125 | 62.5-125 |
| (spores) | | | | | | | | | | |
| B. anthracis | >250 | >250 | 15.6-31.3 | 31.3-62.5 | >250 | >250 | 15.6-31.3 | 31.3-62.5 | >250 | >250 |
| (exponential) | | | | | | | | | | |
| B. cepacia | >250 | >250 | 15.6-31.3 | 31.3-62.5 | >250 | >250 | 15.6-31.3 | 31.3-62.5 | >250 | >250 |
| E. coli | 15.6-31.3 | 31.3-62.5 | 7.8-15.6 | 7.8-15.6 | 31.3-62.5 | 62.5-125 | 7.8-15.6 | 15.6-31.3 | 15.6-31.3 | 15.6-31.3 |

The antimicrobial activity of three AMPs were determined in different assays against other Staphylococcus aureus (NC131142) and the yeast Candida albicans (SC5314). Peptides F11, F11 acetylated at the N-terminus; H04 acetylated at the N-terminus; and MAF-23 (the C-terminal 23 amino acids of LL-37 including MA N-terminal to the FKR sequence); MAF-23 acetylated at the N-terminus and an irrelevant peptide which was not an AMP were synthesised using a standard Fmoc/tBu protocol on an Intavis MultiPep RS (Intavis, Koeln, Germany). Peptide chains were elongated on a tentagel resin (Intavis, Koeln, Germany) using standard protecting groups for the side chains and following the manufacturer's protocols. After TFA cleavage and t-butylmethylether precipitation of the peptides, purification was achieved using an RP-HPLC and a C18 column. The identity of the peptides was confirmed by MALDI-MS and the purity assessed by analytical RP-HPLC. All peptides were found to be >95% pure. Liquid bacterial and yeast cultures were grown overnight in Muller Hinton Broth (MHB) (Oxoid, Basingstoke, UK) for S. aureus and Sabouraud Dextrose Broth (SDB) (Oxoid, Basingstoke, UK) for C. albicans and diluted to approximately 2×10⁶ cfu/ml for S. aureus and 4×10⁴ for C. albicans as measured by plating out onto Muller Hinton Agar (MHB containing 1.5% bacteriological agar) for S. aureus and Sabouraud Dextrose Agar (SDB containing 1.5% bacteriological agar) for C. albicans. Peptides were serially diluted in 25 μl of the appropriate media for the organism and incubated with an equal volume of bacterial or C. albicans culture and grown at 30° C. for 14 hours. Following incubation, the growth of the culture was measured by reading the absorbance of the culture at 595 nm. The absorbance at 595 nm corresponds to the turbity of the culture and therefore the growth.

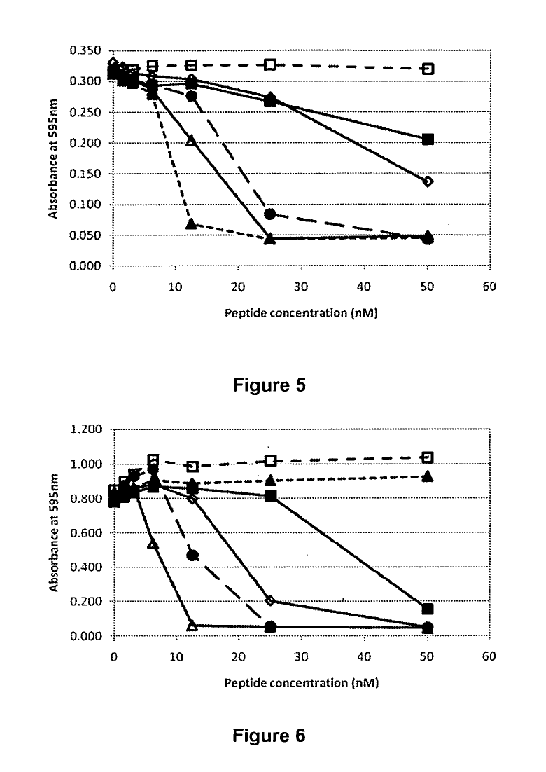

Peptide F11, N-terminally acetylated F11 and N-terminally acetylated H04 all killed S. aureus more effectively than the MA23 or N-terminally acetylated MA23 (FIG. 5). No colonies were recovered at 25 nM for acetylated F11 and approximately 10³ colonies/ml for F11 at this concentration. No colonies were recovered for acetylated H04 at 50 nM. Peptides F11 and acetylated H04 also killed C. albicans more effectively than the wild type peptides MAF23 and acetylated MAF23 (FIG. 6).

### i. Preparation of EEV and IMV-Enriched Virus

Extracellular enveloped virus (EEV)-enriched virus was prepared by synchronously infecting a freshly-confluent monolayer of RK13 cells in a T-25 tissue-culture flask (Falcon), a multiplicity of ≧10. After incubation at room temperature for 30-60 minutes with continuous rocking, the inoculum was removed and replaced with 10 ml of fresh culture medium. The cultures were incubated for 16-24 hours at 37° C. in a 5% CO₂, humidified atmosphere. After the incubation, the culture was observed under a microscope to confirm 100% cytopathic effect (CPE), after which the culture supernatant was carefully removed by pipetting, taking care not to disturb the cell monolayer. The supernate was subjected to room temperature centrifugation at 2500 rpm in a bench-top centrifuge for 5 minutes, after which the supernate was transferred to a clean tube and the pellet discarded. The supernate was taken as being EEV-enriched, and was used fresh on the day of preparation, being maintained at 37° C. until use.

After removal of the EEV-enriched supernate from the cell monolayer, 5 ml of fresh culture medium was added to the T-25 flask, and the flask then frozen at −20° C. in a horizontal position to rupture cell membranes and release intracellular mature virus (IMV) particles. After thawing, the medium was removed and then treated as for preparation of EEV, to give a corresponding IMV-enriched preparation that was used immediately.

### j. Peptide Treatment of Virus Preparations

Orthopoxviruses, of which vaccinia virus is the type member, are a genus of the family poxyiridae, characterised by the production of two morphologically distinct forms of infectious virion. The enveloped virus (EV) form is produced first in the infectious cycle, and is further subdivided to include two mature, morphologically similar types: extracellular enveloped virus (EEV), which is released from the infected-cell surface; and cell-associated enveloped virus (CEV), which is retained on the outer surface of the infected-cell plasma membrane. EV is required for dissemination of an infection in vivo and in vitro. The intracellular mature virus (IMV) form has one less membrane relative to EV and is retained within the infected cell until the cell dies. Its likely function is thought to be associated with maintenance of viability of the virus in the environment, and spread between hosts.

For assays, peptides were diluted to a working stock of 10× final desired concentration in PBS, and 5 μl aliquots dispensed into triplicate screw-cap microcentrifuge tubes. Negative control tubes received 5 μl of PBS. For treatment in the presence of tween-20, an additional aliquot of 5 μl 5% (w/v) tween-20 in H₂O, or 5 μl of H₂O was dispensed into appropriate triplicate tubes. Finally, the prepared EEV- or IMV-enriched virus was dispensed into appropriate triplicate tubes in 40 or 45 μl volumes, to give a final volume of 50 μl per tube. The tubes were capped, mixed by vortexing briefly, and incubated for 1 hour at 37° C. After incubation, 1.5 ml of PBS was added to each tube, mixed by vortexing, and the tubes subjected to centrifugation in a bench-top microcentrifuge for 10 minutes at ≧13,000 g. After centrifugation, the supernate was carefully removed with a 1 ml finnpipette and discarded. A further 1.5 ml of PBS was added to each tube, the tubes mixed by vortexing, and the centrifugation repeated. After the second centrifugation, the supernate was again discarded, and the invisible pellet resuspended in 1.0 ml of tissue-culture medium. The viral suspensions were separately transferred to a safe-break potter-elvejhem homogeniser (Wheaton) and subjected to 5 manual strokes, after which they were transferred to a polystyrene bijou tube.

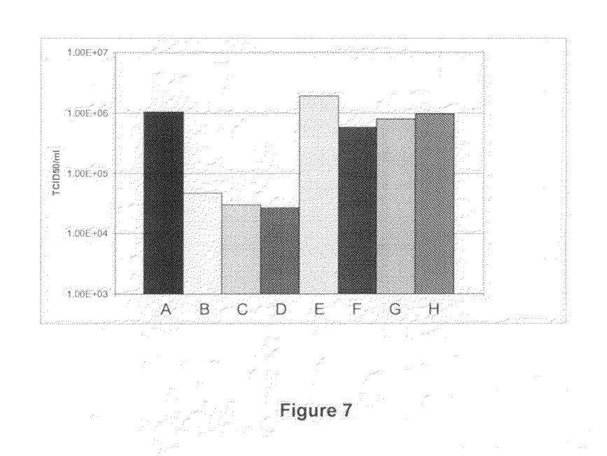

LL-37, F11 and H04 were tested against Vaccinia virus and both AMPs of the invention (i.e. F11 and H04) were demonstrated to have greater anti-viral activity than the wild-type peptide, LL-37 (see FIG. 7). Whether the residual activity following treatment with AMPs was due to small quantities of IMV in the viral preparations was tested with subsequent treatment with an IMV-neutralising serum (RIMV). This resulted in a further decrease in the quantity of viable virus in the preparations by 90 to 99%, indicating that the residual viable virus after treatment with peptides is composed largely of IMV (FIG. 7).

### k. Quantification of Virus

The virus suspension was dispensed in 100 μl volumes in each well of the first column of a sterile tissue-culture 96-well microtitre plate, into which 100 μl of tissue-culture medium had previously been dispensed. The remaining columns were filled with 200 μl/well of culture medium. The virus aliquots were serially diluted across the remaining columns of the plate in steps of 1:3, transferring 100 μl volumes. A 100 μl volume of each serial dilution was transferred to a replica plate containing freshly confluent monolayers of RK13 cells. Cultures were maintained at 37° C. in a 7% CO₂, humidified atmosphere for 6 to 7 days before fixing with 1% formaldehyde and staining with crystal violet. TCID50 (i.e. 50% Tissue Culture Infectious Dose) was calculated by Reed-Muench analysis of virus positive wells.

### l. Sequencing and Sequence Analysis

Positive clones were sequenced by dideoxynucleotide chain termination sequencing methods by Cogenics Ltd (Takeley, UK). The DNA sequences were translated and aligned using Clone Manager Suite 8 (Sci-Ed Software, Cary, N.C., USA) and Bioedit V7.0.5.3. (Ibis Biosciences, Carlsbad, Calif., USA) software packages.

### m. Bacterial Strains, Growth Conditions and Media

Bacterial strains used to test the antimicrobial activity of the AMPs were obtained from the culture collection at the Defence Science and Technology Laboratory (DstI, Porton Down, Salisbury, UK) except S. aureus and C. albicans which were obtained from the Health Protection Agency (London, UK) and ATCC (LGC, Middlesex, UK), respectively. All strains were handled in Advisory Committee for Dangerous Pathogens (ADCP) II containment facilities and were maintained on Luria-Bertoni (LB) agar plates, MHB agar plates or SDB agar plates or broth, or in CDM (Chamberlain's defined media). Strains were grown at 37° C. except for S. aureus and C. albicans which were assayed at 30° C.

### n. Results and Conclusions

The peptide libraries were based upon the wild-type sequence of LL-37 or upon the sequence of a shortened LL-37 motif, KR-20 (nomenclature derived from the N-terminal peptide sequence KR and being 20 amino acids in length), that is also known to have bactericidal activity. Two libraries were designed as described above: (i) the first mutated the LL-37 motif so that, in most instances, 85% of each base in the LL-37 DNA sequence was retained, but 15% of the time it was replaced by one of the other 3 bases; (ii) in the second library, the KR-20 sequence was mutated so that 85% of each base was retained. In the LL-37 doped library sequence Glu-16 and Glu-36 were biased towards Lys, and in the KR-20 doped sequence Glu-36 was biased towards Lys so as to increase the cationic charge of the resultant peptide as it was thought that this would enhance the antimicrobial activity. Glutamic acid was encoded in approximately 6% of the codons at these positions.

To select peptides for stability, whilst maintaining bactericidal activity, a number of interlinked steps were used. First, it was necessary to develop a method for selecting peptides that was capable of binding and causing perturbations in the desired bacterial membranes so that these peptides could be enriched by a binding event in the selection. In one experiment, Bacillus subtilis membranes were prepared by rupturing the bacteria using a pressure cell, followed by coating the membranes onto polystyrene magnetic beads. Four rounds of selection were performed and then screened. The membrane preparation was tested by coating the membranes onto MaxiSorp™ polystyrene plates and wild-type full length LL-37 peptide fused to 3×FLAG-10×His was used as a control to demonstrate significant binding to the immobilised Bacillus subtilis membranes. Treatment of the expressed peptide with either subtilisin or V8 protease demonstrated the lability of this peptide to either of the proteases and, thus, abolished the signal associated with binding of the peptide to the membranes. This also demonstrated the suitability of this assay for determining the proteolytic resistance of LL-37 mutants expressed in this format.

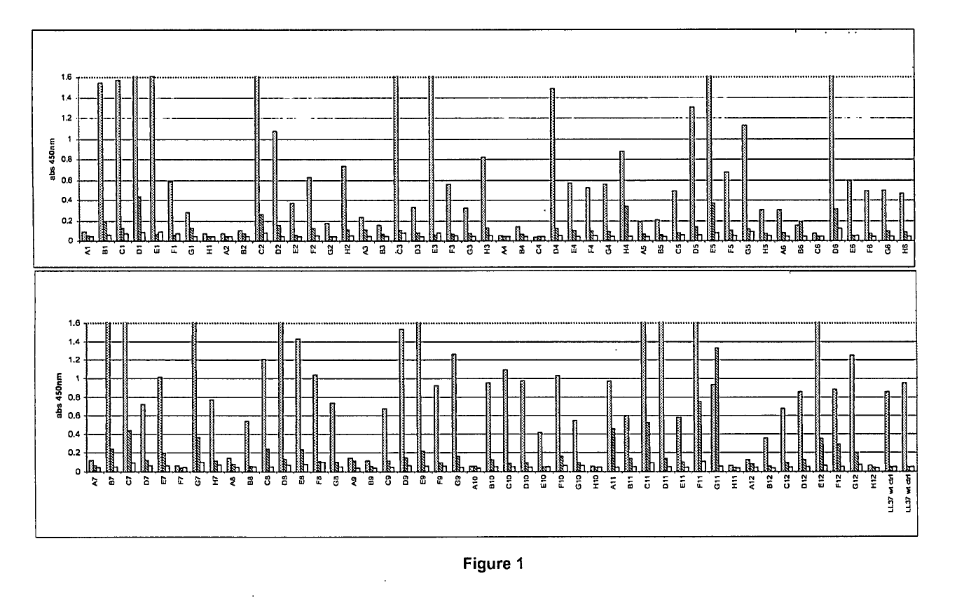

Significant numbers of clones selected from the pool of binders showed some degree of resistance to the subtilisin and V8 protease cocktail, as indicated by the preservation of a signal after treatment with protease (FIG. 1). Protease treatment of G11 appears to increase signal strength of this clone under these assay conditions. The clones were sequenced and aligned: a selection of particularly useful peptide sequences is shown in Table 3. The expected mutation rate for most codons was 39% (1-0.85³) but it was apparent, in library 1, that the mutation rate was lower between residues 10-27 than for the rest of the peptide. This was also lower than the expected value (data not shown), and correlates with the proposed antibacterial activity of the peptide, which is believed to encompass residues 11-32 (Strömstedt et al., 2009). Thus, the results indicated that the membrane-binding assay was a positive indicator of antibacterial activity.

When the sequences of the stabilised peptides from library 1 were compared, certain positions were invariant (Arg19, Arg23, Lys25, Leu31 and Val32). It is not immediately apparent what contribution these amino acid side chains are making towards the stability of the peptide. In library 2, again it was clear that the mutation rate between residues corresponding to 18-32 in LL-37 was far below the expected value, while the amino acids at the C-terminus of the peptide were less conserved. This also correlates with the known solution structures of LL-37 bound to SDS micelles in which F17-R29 is helical and the C-terminus of the peptide is more disordered (Li et al., 2005).

In order to establish that the stabilised peptides retained useful antimicrobial properties, the peptides C11 from library 1; and G11, E11 and H04 from library 2; were synthesised and tested in antimicrobial assays alongside wild-type peptides. The wild-type peptides used in these assays were LL-37 (wild-type); and LL-37 and KR-20 with MA (met-ala) N-terminal extensions, termed MA-39 and KR-22, respectively. The sequence MA-39 was a useful control for the addition of MA- on the peptides selected from the library (a result of cloning).

TABLE 3
Sequences of protease resistant binding clones from round
4 were aligned for similarity (PAM250). Clones are shown
below the libraries from which they were selected.
Residues identical to wild-type are shaded while mutations
are unshaded. SEQ ID NO: 3 corresponds to the 21 C-terminal
amino acids of SEQ ID NO: 2.
Library 1: Full length library clones

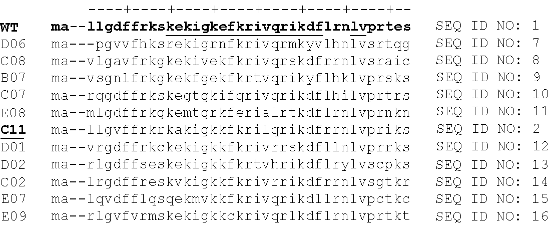

Library 2: Truncation library clones

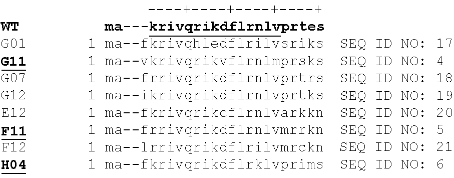

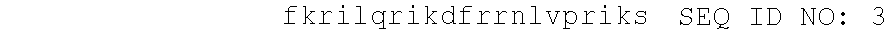

The antimicrobial assays were initially performed over a time course of up to 24 hours against Bacillus anthracis in order to assay for prolonged activity against the bacteria either as spores (FIG. 2) or in a vegetative stage (FIG. 3). The results show that the wild-type MA-22 peptide has weak activity against the spores and no activity against the vegetative cells. Wild-type LL-37 and MA-39 both had better activity than MA-22 against Bacillus anthracis spores, but this activity diminished rapidly over time, so that activity was lost within the 24 hour period of the test. LL-37 and MA-39 did not have activity against vegetative cells, which helps to highlight the sensitivity of LL-37 to proteolytic degradation.

The stabilised peptides C11, G11, F11 and H04 that were tested had improved bactericidal activity against the Bacillus anthracis spores when compared to MA-22, MA-39 and LL-37. The duration of antibacterial activity was particularly extended for peptides F11 and H04, which were still effective at the end of the 24-hour period in both spore and vegetative cell assays. It is worth noting that the net charge of the peptides was increased over wild type in that MA-22 has a net charge of +4, whereas G11, F11 and H04 have net charges of +7, +8 and +6, respectively. LL-37 has a net charge of +6 whereas C11 has a net charge of +14. These data suggest that an increase in the cationic charge of the AMPs (relative to corresponding wild-type sequences) may be useful for enhancing antimicrobial activity, particularly antibacterial activity.

In addition, the peptides were further tested against different bacterial species including Burkholderia cepacia, that is known to cause rapid decline in lung function, which can result in severe lung disease and may even lead to death in a small population of cystic fibrosis patients. Peptides F11 and H04 were tested for bactericidal activity against Burkholderia cepacia and were found to exhibit greater killing capacity than LL-37. Furthermore, both tested peptides demonstrated effective killing activity over the entire duration of the 24-hour testing period (FIG. 4). Significantly, since native LL-37 had no effect on the growth of B. cepacia, these data demonstrate both the greater efficacy of AMPs of the invention compared to wild-type peptides and the potential for broader spectrum antimicrobial activity.

In comparable tests, the selected peptides displayed antibacterial activity against Francisella tularensis (data not shown) and E. coli (Table 2). Peptides F11 and H04 demonstrate better activity than wild type LL-37 in assays against B. anthracis, B. cepacia and E. coli and improved sporicidal activity against B. anthracis spores. The improved activity and efficacy of the AMPs of the invention is still more pronounced when it is considered that the corresponding KR-20 peptide of LL-37 is generally less effective than LL-37 in the present studies.

In antimicrobial tests against S. aureus and C. albicans, peptides F11, N-terminally acetylated F11 and N-terminally acetylated H04 were tested against the shortened peptide derivative of LL-37, MAF-23. All these peptides were C-terminally amidated. LL-37 was not tested in these assays. The selected peptides had greater killing effect than MAF-23 wild type derivatives, MAF-23 and acetylated MAF-23, against S. aureus and C. albicans except acetylated F11 which had less activity than the MAF-23 peptides. The acetylated peptide F11 lacked activity in this assay, which may be a result of the modification of the charge at the N-terminus, as unmodified F11 possessed greater activity than wild type (FIGS. 5 and 6).

In summary, these tests demonstrate that the selected peptides have advantageous antibacterial activity against both Gram-positive and Gram-negative bacteria.

Furthermore, the results demonstrate that the methods of the invention can be used to synthesise and select for AMPs that have prolonged antimicrobial activity, for example, as a result of increased protease resistance compared to wild-type antimicrobial peptides and proteins.

Finally, the selected peptides were assayed for anti-viral activity against Vaccinia virus. The results demonstrate that the peptides retained the virucidal potency of wild-type LL-37 against Vaccinia virus (FIG. 7). The activity of the peptides against EEV-enriched preparations did not result in complete inactivation of all virus in these samples. However, it was thought that this may be due to small quantities of IMV in the EEV-enriched preparations, and this possibility was examined by treatment of the virus preparation with the peptides and subsequent treatment with an IMV-neutralising serum (RIMV). Subsequent treatment of EEV-enriched preparations with RIMV after incubation with peptide resulted in a further decrease in the quantity of viable virus in the preparations by between 90 and 99%, which indicated that the residual viable virus after initial treatment with AMPs of the invention is composed largely of IMV. This suggests that the peptides strip the outer layer of the EEV form allowing the neutralisation serum to neutralise the IMV form.

Therefore, the selected AMPs of the invention have a broad range of antimicrobial activity, against viruses and bacterial, which is equivalent to or better than known wild-type antimicrobial peptides. It may also be an advantage in applications involving the AMPs of the invention (e.g. in therapy), that AMPs of reduced amino acid length compared to wild-type peptide sequences (such as LL-37) can be used effectively, and in many cases, they have even greater antimicrobial activity and greater protease resistance than the corresponding wild-type sequences. The method of the invention is, thus, particularly beneficial in producing specialised AMPs having desirable and specific characteristics of protease resistance and antimicrobial activity for particular applications.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1-49. (canceled)

50. An antimicrobial peptide (AMP) comprising at least 12 consecutive amino acid residues of a mutated sequence of LL-37, and wherein the AMP comprises at least 3 amino acid substitutions of the sequence of LL-37 (SEQ ID NO: 1: LLGDFFRKSKEKIGKEFKRIVQRIKDFLRNLVPRTES) selected from the group consisting of: Ser37 to Asn; Glu36 to Lys, Arg or Met; Thr35 to Ile, Ser, Lys, Arg or Cys; Pro33 to Ser, Met or Ala; Val32 to Met; Asn30 to Lys or Ile; Leu28 to Arg; Asp26 to Val or Cys; Val21 to Leu; Lys 18 to Arg; Phe17 to Val, Ile or Leu; Glu16 to Lys; Glu11 to Ala; Ser9 to Arg; and Asp4 to Val.

51. The AMP of claim 50, wherein the AMP includes at least 3 substitutions of the sequence of SEQ ID NO: 1 selected from: Ser37 to Asn; Glu36 to Lys, Arg or Met; Thr35 to Ile, Ser, Lys, Arg or Cys; Pro33 to Ser, Met or Ala; Val32 to Met; and Asn30 to Lys or Ile.

52. The AMP of claim 50, which comprises at least 18 consecutive amino acids of the sequence:
N′-RIxQRIKxFxRxLxxRxKx-C′
wherein x is any amino acid residue.

53. An AMP of claim 50, which comprises at least 18 consecutive amino acids of the sequence:
N′-RIVQRIKX₁FX₂RX₃LX₄X₅RX₆X₇X₈-C′
wherein X₁ is D, V, C or Y; X₂ is L or R; X₃ is N, I or K; X₄ is V or M; X₅ is P, S, A or M; X₆ is T, K, R, C, I or S; X₇ is E, K, R or M; and X₈ is S or N.

54. The AMP of claim 53, wherein:
(i) X₁ is D or V; X₂ is L or R; X₃ is N or K; X₄ is V or M; X₅ is P, S or M; X₆ is K, R, S or I; X₇ is K, R or M; and X₈ is S or N; or
(ii) X₁ is D; X₂ is L; X₅ is P or M; X₆ is R, S or I; and X₇ is K.

55. The AMP of claim 50, which mutated sequence of LL-37 has higher cationic charge than the corresponding sequence of SEQ ID NO: 1.

56. The AMP of claim 50, which comprises between 12 and 50 amino acids; between 18 and 40 amino acids; between 20 and 37 amino acids; or between 20 and 24 amino acids.

57. The AMP of claim 50, comprising between 18 and 37 consecutive amino acids of a mutated sequence of LL-37, and wherein the mutated sequence of LL-37 comprises at least the following sets of amino acid substitutions:
(i) Asp4 to Val, Ser9 to Arg, Glu11 to Ala, Glu16 to Lys, Val21 to Ile, Leu28 to Arg, Thr35 to Ile and Glu36 to Lys;
(ii) Phe17 to Val, Asp26 to Val, Val32 to Met, Thr35 to Ser and Glu36 to Lys;
(iii) Lys18 to Arg, Pro33 to Met, Thr35 to Arg, Glu36 to Lys and Ser37 to Asn; or
(iv) Asn30 to Lys, Thr35 to Ile and Glu36 to Met.

58. The AMP of claim 50, which comprises a sequence selected from:

| | |
|---|---|
| (i) | LLGVFFRKRKAKIGKKFKRILQRIKDFRRNLVPRIKS; |
| (ii) | FKRILQRIKDFRRNLVPRIKS; |
| (iii) | VKRIVQRIKVFLRNLMPRSKS; |
| (iv) | FRRIVQRIKDFLRNLVMRRKN; |
| and | |
| (v) | FKRIVQRIKDFLRKLVPRIMS. |

59. The AMP of claim 50, which is amidated at the C-terminus.

60. The AMP of claim 50 that has an intramolecular, non peptide bond.

61. The AMP of claim 50, which is conjugated to another peptide moiety, or a non-peptide moiety.

62. A nucleic acid encoding the AMP amino acid sequence of claim 50.

63. A pharmaceutical composition comprising the AMP of claim 50.

64. The composition of claim 63, formulated for topical application.

65. The composition of claim 64, which is formulated as a handwash and which is a sporicidal or bacteriocidal composition.

66. A method for treating a microbial infection in a subject, the method comprising administering an effective amount of an AMP according to claim 50.

67. The method of claim 66, wherein the microbial infection is selected from bacteria, fungi, parasites and enveloped viruses; or wherein the microbial infection is a bacteria selected from the group consisting of Bacillus anthracis, Burkholderia cepacia, Clostridium difficile, Staphlococcus aureus, Streptococcus mutans, Pseudomonas aeruginosa, Mycobacterium tuberculosis, Escherichia coli and Francisella tularensis.

68. The method of claim 67, wherein Bacillus anthracis exist as spores.

69. A method for treating a cancer in a subject, the method comprising administering an effective amount of an AMP according to claim 50.
